(19) United States
(12) Patent Application Publication
(10) Pub. No.: US 2002/0194639 A1
(43) Pub. Date: Dec. 19, 2002

(54) Bonsai, a phospholipid binding protein, is required for thermal tolerance in arabidopsis

(76) Inventors: Jian Hua, Cambridge, MA (US); Paula Grisafi, Sudbury, MA (US); Gerald R. Fink, Chestnut Hill, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 09/891,139

(22) Filed: Jun. 25, 2001

Related U.S. Application Data
(60) Provisional application No. 60/213,863, filed on Jun. 23, 2000.

(51) Int. Cl. A01H 5/00; C07H 21/04; C12N 9/24

(52) U.S. Cl. 800/278; 435/419; 435/200; 435/320.1; 536/23.2

(57) ABSTRACT

The invention relates to genes and proteins, namely, BON1, BON2, BON3, BAP1 and BAL, the expression of which modulate the size of a plant, and transgenic plants comprising those genes and proteins, and method of making and using same.

### RELATED APPLICATION

This application claims the benefit of U.S. Provisional Application No. 60/213,863, filed on Jun. 23, 2000, the entire teachings of the which are incorporated herein by reference.

### GOVERNMENT SUPPORT

The invention was supported, in whole or in part, by grant MCB-9974451 from the National Science Foundation. The Government has certain rights in the invention.

### BACKGROUND OF THE INVENTION

Multicellular organisms develop and maintain a relatively constant size and shape over a wide range of different environmental conditions. This homeostasis is accomplished both by extrinsic mechanisms, e.g., movement that can reestablish a constant environment, and also by intrinsic mechanisms, e.g., mechanisms that alter cellular metabolism so that the organism can retain its morphology despite the altered environment. Animals have the ability to relocate themselves to a different location to maintain constant external conditions, whereas most plants are anchored and can only bend in response to changes in environmental factors such as light, gravity, or mechanical force. and shape in response to an environmental perturbation. Recently, a genetic pathway has been discovered in Drosophila that regulates cell, organ and body size. Mutations in genes apparently functioning in the insulin-signaling pathway result in miniature flies comprised of smaller and fewer cells under conditions where wild type flies maintain their standard size (Oldham, S., R. Bohni et al. (2000) Philos. Trans. R. Soc. Lond. B Biol. Sci. 355(1399):945-52). These mutant flies phenocopy the wild type flies under conditions of starvation or overcrowding, which reveals a link between the control of morphology and nutritional conditions that exist in the environment.

For plants, these intrinsic genetic mechanisms must be essential in temperature homeostasis because these organisms are sessile and do not maintain constant body temperatures when the ambient temperature changes. Indeed, most plants maintain a relatively constant phenotype in varied temperatures. For example, Arabidopsis grows to similar size and morphology (less than 2-fold change) over temperatures that range from 16° C. to 30° C. This suggests the existence of genes whose function is to permit adaptation to temperature variation.

Studies on the tolerance of plants to freezing have revealed the mechanisms that contribute to survival at very low temperatures. Both physiological and genetic analyses point to the membrane systems as important to this homeostasis. Extreme cold results in the disruption of membrane function and death (Steponkus, P. L. (1984) Annu. Rev. Plant Physiol. 35:543-84). Plants can increase their tolerance to extreme cold by prior exposure to low, but non-freezing temperatures, a phenomenon known as cold acclimation. A prominent feature of cold acclimation is an alteration of the membrane lipid composition and the accumulation of simple sugars and some hydrophilic peptides, both of which are thought to stabilize membranes to protect against freezing injury (Thomashow, M. F. (1999) Annu. Rev. Plant Physiol. 50:5,71-99). Genetic analysis has implicated the plasma membrane as important to thermotolerance. Mutants hypersensitive to freezing injury (e.g., sfr) have defects in the cryostability of the plasma membranes (Warren, G., R. McKown et al. (1996) Plant Physiol 111(4):1011-9).

A need exists to further define the genetic control of homeostasis in plants. Plants in which size can be manipulated would be very useful commercially, in both the agricultural and ornamental plant markets.

### SUMMARY OF THE INVENTION

Wild-type Arabidopsis plants maintain a relatively constant size over a wide range of temperatures. This homeostasis requires the BONZAI1 (BON1) gene because bon1 null mutants produce miniature fertile plants at 22° C., but produce a wild-type phenotype when grown at 28° C. The expression of BON1 and a BON1-associated protein (BAP1) is modulated by temperature. Thus, BON1 and BAP1 have a direct role in regulating cell expansion and cell division at lower temperatures. BON1 contains a Ca²⁺-dependent phospholipid-binding domain and is associated with the plasma membrane. It belongs to the copine gene family, which is conserved from protozoa to humans. This gene family may function in the pathway of membrane trafficking in response to external conditions.

As disclosed herein, the BONZAI1 (BON1), BON2, and BON3 genes permit wild-type Arabidopsis plants to maintain a relatively constant size over a wide range of temperatures. bon1 null mutants produce miniature fertile plants at 22° C., but produce a wild-type phenotype when grown at 28° C. A protein associated with BON1, “BON1associated protein”, or BAP1, is also modulated by temperature. Thus, BON1 and BAP1 have a direct role in regulating cell expansion and cell division at temperatures lower than those at which Arabidopsis is normally grown. BON1 contains a Ca²⁺ dependent phospholipid-binding domain and is associated with the plasma membrane, and belongs to the copine gene family, which is conserved from protozoa to humans.

In one embodiment, the present invention features an isolated nucleic acid comprising SEQ ID NO:2, and degenerate variants thereof. The invention also features an isolated nucleic acid comprising 315 or more consecutive nucleotides of SEQ ID NO:2, and also an isolated nucleic acid comprising a sequence that encodes a polypeptide having an amino acid sequence which is 94% identical to SEQ ID NO:3. The invention also features an isolated polypeptide comprising the amino acid sequence of SEQ ID NO:3, and also an isolated polypeptide comprising 400 or more consecutive amino acid residues of SEQ ID NO:3, and also an isolated polypeptide comprising a sequence that has 94% sequence identity to SEQ ID NO:3.

In another embodiment, the present invention features a vector comprising the isolated nucleic acids of SEQ ID NO:2 or degenerate variants thereof, and isolated cells (e.g., a prokaryotic cell, a eukaryotic cell) containing such a vector.

In another embodiment, the present invention features a transgenic plant which has an altered size compared to a corresponding non-transgenic plant, comprising introducing into the plant exogenous nucleic acid which modulates BON1 in the plant. The transgenic plant can be smaller in size. In one embodiment, the invention features a transgenic plant that is smaller in size than a corresponding non-transgenic plant, comprising introducing exogenous nucleic acid which inhibits BON1 in the plant. The exogenous nucleic acid can be inserted in the twelfth exon of the BON1 gene. The exogenous nucleic acid can be a fusion of BON1 with a beta-glucuronidase gene. The exogenous nucleic acid can be inserted in the second exon of the BON1 gene.

In another embodiment, the invention also features a transgenic tissue culture derived from the transgenic plant as described above, and a transgenic seed of such a transgenic plant, and a transgenic plant, plant part, plant cell or tissue culture, grown from such a transgenic seed. The transgenic plant, plant part, plant cell or tissue culture can be an ornamental plant or a turfgrass, or the plant part, plant cell or tissue culture can be derived from an ornamental plant or a turfgrass.

In another embodiment, the invention features a transgenic plant that is larger in size than a corresponding non-transgenic plant, comprising introducing into the plant an exogenous nucleic acid which enhances BON1 in the plant. The the exogenous nucleic acid can be an exogenous BON1 gene.

The invention also features a method of producing a transgenic plant which has an altered size compared to a corresponding non-transgenic plant, comprising introducing into the plant exogenous nucleic acid which modulates BON1 in the plant.

The invention also features a method of producing a transgenic plant that is smaller in size than a corresponding non-transgenic plant, comprising introducing into the plant an exogenous nucleic acid which inhibits BON1 in the plant. The method can be accomplished by mutating the endogenous BON1 in the plant. The exogenous nucleic acid can be a fusion of BON1 with a beta-glucuronidase gene (BON1-GUS). The exogenous nucleic acid can result in overexpression of the C-terminus of a BON1, or overexpression of the full length BON1. The transgenic plant can be an angiosperm or a gymnosperm. The transgenic plant can be an ornamental plant or a turfgrass.

The invention also features a method of producing a transgenic plant that is larger in size than a corresponding non-transgenic plant, comprising introducing into the plant an exogenous nucleic acid which enhances BON1 in the plant. The exogenous nucleic acid can be an exogenous BON1 gene. The plant can be a crop plant, or a plant grown and harvested for its biomass.

In another embodiment, the invention also features a method of modulating homeostasis of a plant, the method comprising introducing into the plant an exogenous nucleic acid which modulates BON1 in the plant.

The present invention relates to a method to increase the yield of a plant, comprising introducing an exogenous nucleic acid that enhances BON1 in the plant. The method can be used to produce a plant that can grow at a higher altitude or in a lower temperature region than a corresponding non-transgenic plant, where the method comprises introducing into the plant an exogenous nucleic acid that enhances BON1 in the plant.

In another embodiment, the invention features an isolated nucleic acid comprising SEQ ID NO:5, and degenerate variants thereof. The nucleic acid can comprise 170 or more consecutive nucleotides of SEQ ID NO:5. The nucleic acid can comprise a sequence that encodes a polypeptide having an amino acid sequence which is 97% identical to SEQ ID NO:6.

The invention also features an isolated polypeptide comprising the amino acid sequence of SEQ ID NO:6, and an isolated polypeptide comprising 550 or more consecutive amino acid residues of SEQ ID NO:6, and an isolated polypeptide comprising a sequence that has 97% sequence identity to SEQ ID NO:6.

In another embodiment, the present invention features a vector comprising the isolated nucleic acids of SEQ ID NO:5 or degenerate variants thereof, and isolated cells (e.g., a prokaryotic cell, a eukaryotic cell) containing such a vector.

In another embodiment, the invention features an isolated nucleic acid comprising SEQ ID NO:11, and degenerate variants thereof. The nucleic acid can comprise 390 or more consecutive nucleotides of SEQ ID NO:11. The nucleic acid can comprise a sequence having 99% sequence identity to the coding sequence of SEQ ID NO:11. The nucleic acid can comprise a sequence that encodes a polypeptide having an amino acid sequence which is 98% identical to SEQ ID NO:12.

The invention also features an isolated polypeptide comprising the amino acid sequence of SEQ ID NO:12, and an isolated polypeptide comprising 180 or more consecutive amino acid residues of SEQ ID NO:12, and an isolated polypeptide comprising a sequence that has 97% sequence identity to SEQ ID NO:12.

In another embodiment, the present invention features a vector comprising the isolated nucleic acids of SEQ ID NO:11 or degenerate variants thereof, and isolated cells (e.g., a prokaryotic cell, a eukaryotic cell) containing such a vector.

In another embodiment, the present invention features a method of producing a transgenic plant which has an altered size compared to a corresponding non-transgenic plant, comprising introducing into the plant exogenous nucleic acid which modulates BAP1 in the plant. In one embodiment, the method is a method of producing a transgenic plant that is smaller in size than a corresponding non-transgenic plant comprising introducing into the plant an exogenous nucleic acid sequence which inhibits BAP1. The method can be accomplished by mutating the endogenous BAP1 in the plant. The transgenic plant can be an angiosperm or a gymnosperm. The transgenic plant can be an ornamental plant or a turfgrass.

In another embodiment, the invention also features a transgenic tissue culture derived from the transgenic plant as described above, and a transgenic seed of such a transgenic plant, and a transgenic plant, plant part, plant cell or tissue culture, grown from such a transgenic seed. The transgenic plant, plant part, plant cell or tissue culture can be an ornamental plant or a turfgrass, or the plant part, plant cell or tissue culture can be derived from an ornamental plant or a turfgrass.

The invention also features a method of producing a transgenic plant that is larger in size than a corresponding non-transgenic plant, comprising introducing into the plant an exogenous nucleic acid which enhances BAP1 in the plant. The exogenous nucleic acid can be an exogenous BAP1 gene. The plant can be a crop plant, or a plant grown and harvested for its biomass.

In another embodiment, the invention also features a method of modulating homeostasis of a plant, the method comprising introducing into the plant an exogenous nucleic acid which modulates BAP1 in the plant. The method can be used to increase the yield of a plant. The method can be used to produce a plant that can grow at a higher altitude or in a lower temperature region than a corresponding non-transgenic plant, where the method comprises introducing into the plant an isolated nucleic acid that enhances BAP1 in the plant.

In another embodiment, the invention also features a transgenic plant produced by any of the methods described herein.

### BRIEF DESCRIPTION OF THE DRAWINGS

FIGS. 1A, 1B, 1C, 1D, 1E and 1F are a set of six photographs showing the phenotype of bon1-1 plants.

FIG. 1A shows wild type Arabidopsis (Col-0) and bon1-1 grown at 22° C.

FIG. 1B shows wild type Arabidopsis (Col.-0) and bon1-1 grown at 28° C.

FIG. 1C shows leaves of wild type Arabidopsis (Col-0) and bon1-1 grown at 22° C.

FIG. 1D shows stems of wild type Arabidopsis (Col-0.0) and bon1-1 grown at 22° C.

FIG. 1E shows wild type Arabidopsis (Col-0) and bon1-1 grown at 22° C. for three weeks and then transferred to 28° C. for ten days.

FIG. 1F shows wild type Arabidopsis (Col-0) and bon1-1 grown at 28° C. for three weeks and then transferred to 22° C. for ten days.

FIGS. 2A and 2B are a set of two diagrams illustrating the structure of the BON1 gene (FIG. 2A) and the BON1 protein (FIG. 2B).

FIG. 2A shows the genomic structure of the BON1 gene. Exons are represented by boxes, and translation start and stop are indicated by “ATG” and “STOP”, respectively. The T-DNA insertion sites in bon1-1 and bon1-2 are in exons 12 and 2 as indicated.

FIG. 2B shows the domains of the BON1 protein, which has two C2 domains at the N-terminus and an A domain at the C-terminus.

FIG. 3 is a diagram showing the alignment of the amino acid sequences for Arabidopsis thaliana BON1 (SEQ ID NO:3), BON2 (SEQ ID NO:6) and BON3 (SEQ ID NO:9) proteins, aligned with the COPINE1 protein (SEQ ID NO:16) from Homo sapiens.

FIGS. 4A, 4B, 4C and 4D show BON1 expression.

FIGS. 4A, 4B and 4C are a set of three photographs showing the BON1-GUS expression pattern. Dark blue staining indicates GUS expression.

FIG. 4A shows expression in seedlings at the 4-leaf stage; expression is seen in young leaves.

FIG. 4B shows expression in seedlings before bolting; expression is seen in younger leaves and not in older ones.

FIG. 4C shows expression in the inflorescence stem; expression is seen in the apical portion of the stem.

FIG. 4D is a Northern blot analysis showing that BON1 RNA expression is modulated by temperature. RNA samples were isolated from 5-week-old plants grown at 28° C. (lane 1), shifted from 28° C. to 22° C. for 12 hours (lane 2), grown at 22° C. (lane 3), shifted from 22° C. to 28° C. for 12 hours (lane 4).

FIGS. 5A, 5B, 5C and 5D show the association of BON1 with membranes.

FIGS. 5A, 5B, 5C and 5D are photographs of SDS-PAGE gels stained with Coomassie Blue.

FIG. 5A shows that BON1 binds lipid. BON1 protein was mixed with (+) or without (−) phosphotidyl serine (PS) or calcium ion. The mixture was spun down and the protein in the pellet was run on SDS-PAGE and stained with Comassie Blue.

FIG. 5B shows that BON1 is associated with membranes in the plants. Total proteins from BON1-HA plants were extracted and incubated with buffer, buffer with 0.5 M NaCl, 2.5 M urea, 0.1 M Na₂CO₃, 1% Triton X-100, and 1% sarcosyl, respectively, for one hour. The soluble and pellet fractions were separated by ultracentrifugation. Total protein, soluble fractions, and pellet fractions were then separated by SDS-PAGE, blotted, and probed with anti-HA antibody.

FIG. 5C shows that calcium stimulates the association of BON1 with membranes. Total proteins from BON1-HA plants were incubated with buffer containing EGTA (25 mM) or calcium ion (25 mM) before fractionation by centrifugation.

FIG. 5D shows subcellular fractionation by sucrose density centrifugation. The microsomal fraction from transgenic Arabidopsis containing BON1-HA was separated on a sucrose gradient of 25% to 50%. Proteins from each fraction were separated on a 4-20% SDS-PAGE gel and blotted to membranes. The blots were probed with anti-HA antibody, and antibodies against marker proteins from different membranes: plasma-membrane ATPase (PM-ATPase) for plasma membrane, BIP for endoplasmic reticulum, pyrophosphate (PPase) for vacoules. The activity of Golgi specific UDPase activity was assayed (Schaller, G. E. and N. D. DeWitt (1995) Methods Cell Biol. 50:129-48) in each fraction and the strength of the activity was indicated from very strong (++++) to low (−).

FIG. 6 is a bar graph showing vesicle aggregation promoted by BON1 in vitro. Vesicle aggregation was monitored by the turbidity of the lipid, which was measured by the absorbance at 540 nm. Phosphotidylserine was incubated with no protein (“A”), control protein (“B”), the two C2 domains of BON1 (C2A-C2B) (“C”), or full-length BON1 protein (C2A-C2B-A) (“D”) in the presence (Ca²⁺) or absence (−) of calcium ion for one-half hour.

FIGS. 7A and 7B are a pair of Northern blots showing BAP1 expression and function.

FIG. 7C is a photograph showing suppression of the bon1-1 phenotype by BAP1 overexpression.

FIG. 7A is a Northern blot of total RNAs from different tissues (root, leaf, stem, and flower), showing tissue distribution of BAP1. BON1 and BAP1 show higher expression in leaves and stems and BAP1 also has higher expression in roots.

FIG. 7B shows BAP1 RNA expression in wild type and the bon1-1 mutant grown at 22° C. and 28° C.

FIG. 7C shows suppression of the bon1-1 phenotype by BAP1 overexpression. Wild type and bon1-1 are on the left for comparison. Four independent transgenic lines of 35S::BAP1 in a bon1-1 background are on the right. The plants also show varying degrees of suppression of bon1-1 phenotype by overexpression of BAP1.

FIGS. 8A, 8B and 8C, respectively, show the BON1 genomic (SEQ ID NO:1), BON1 cDNA (SEQ ID NO:2) and BON1 protein (SEQ ID NO:3) sequences.

FIGS. 9A and 9B, respectively, show the BON2 coding (SEQ ID NO:5) and BON2 protein (SEQ ID NO:6) sequences.

FIGS. 10A and 10B, respectively, show the BON3 coding (SEQ ID NO:8) and BON3 protein (SEQ ID NO:9) sequences.

FIGS. 11A and 11B, respectively, show the BAP1 coding (SEQ ID NO: 11) and BAP1 protein (SEQ ID NO:12) sequences.

FIGS. 12A and 12B, respectively, show the BAL coding (SEQ ID NO:14) and BAL protein (SEQ ID NO:15) sequences.

### DETAILED DESCRIPTION OF THE INVENTION

The invention relates to nucleic acids (DNA, cDNA, RNA, mRNA) and proteins which modulate plant growth homeostasis. The nucleic acids and proteins described herein control cell expansion and cell division, resulting in changes in the size and rate at which the host plant grows when exposed to lower temperatures. In particular, the present invention relates to nucleic acids expresing the BONZAI1 protein (BON1), and its homologs BON2 and BON3, the BON1-associated protein (BAP1) and the BAL:BAP-Like protein (BAL).

The Arabidopsis BON1 genomic sequence is presented as SEQ ID NO:1, and the cDNA as SEQ ID NO:2. The predicted BON1 protein sequence is presented as SEQ ID NO:3. Two homologs of the BON1 protein, BON2 and BON3 are also disclosed herein. The BON2 genomic sequence is presented as SEQ ID NO:4, the BON2 coding sequence as SEQ ID NO:5, and the predicted BON2 protein sequence as SEQ 11) NO:6. The BON3 genomic sequence is presented as SEQ ID NO:7, the BON3 coding sequence as SEQ ID NO:8, and the predicted BON2 protein sequence as SEQ ID NO:9.

The Arabidopsis BAP1 genomic sequence is presented as SEQ ID NO:10, and the BAP1 coding sequence as SEQ ID NO:11. The predicted BAP1 protein sequence is presented as SEQ ID NO:12.

A blast search revealed that BAP1 has homology to another putative Arabidopsis protein in the database which, for purposes of this study, was called BAL (“BAP1 Like”). The BAL genomic sequence is presented as SEQ ID NO:13, and the BAL coding sequence as SEQ ID NO:14. The predicted BAL protein sequence is presented as SEQ ID NO:15.

One approach to identifying the genes involved in temperature homeostasis is to isolate Arabidopsis mutants that are unable to maintain size or shape over a broad temperature range. A mutant was isolated using this approach, bonzai1 (bon1), which makes miniature plants at lower temperatures. As described herein, the BON1 gene encodes one of the copines, a protein widely conserved in plants and animals. The BON1 protein is tightly associated with the plasma membrane and promotes aggregation of lipid vesicles in vitro. BON1 associates with another protein BAP1 which, when overexpressed, can suppress the bon1 phenotype. As described herein, the copine gene family may function in membrane trafficking and be transcriptionally regulated by the environmental conditions to which they are designed to respond.

The invention is directed to isolated nucleic acids, BON1, BON2, BON3, BAP1 and BAL, which encode proteins that are necessary for normal growth. Inhibition of one or more of these genes results in plants which are smaller in size (minaturized) when grown at lower temperature, compared to the size of the corresponding wild type plant (the same type of plant as the mutant but which does not have a defect in one or more of these genes) when grown at the same lower temperature. Enhancement of one or more of these genes results in plants which are larger in size, compared to the size of the corresponding wild type plant (the same type of plant as the mutant but which does not have a defect in one or more of these genes).

The present invention also relates to transgenic plants having altered size (smaller in size, larger in size) compared to the size of a corresponding wild type plant, wherein the transgenic plant comprises exogenous nucleic acid which modulates (inhibits, enhances) BON1, BON2, BON3, BAP1 and/or BAL in the transgenic plant. As used herein, “exogenous nucleic acid” is nucleic acid which is obtained from a source other than the recipient plant cell (i.e., the plant cell into which the exogenous nucleic acid is being introduced), and which, when encoding a peptide, is stably expressed. The exogenous nucleic acid can be present episomally or integrated into the genome of the plant cell (into genomic nucleic acid). The exogenous nucleic acid can be DNA, DNA obtained from a source in which it occurs in nature, or produced by synthetic or recombinant methods.

In one embodiment, the present invention relates to a transgenic plant that is smaller in size than a corresponding non-transgenic plant, wherein the plant comprises exogenous nucleic acid which inhibits BON1, BON2, BON3 and/or BAP1 in the plant. The inhibition of BON1, BON2, BON3 or BAP1 can be partial or complete. Because BON1, BON2, BON3 and BAP1 are likely involved in a physiological pathway, inhibiting BON1, BON2, BON3 or BAP1 includes, for example, inhibition of the gene by knocking out the gene or mutating the gene in a way to render it non-functional (e.g., so that the gene does not encode a functional protein). Inhibition also includes interfering with the regulatory region of the gene, e.g., introducing a nucleic acid which prevents expression or the timing of the encoded product (e.g., splicing in a regulatory region, either upstream or downstream of the gene, which negatively regulates expression of the gene (interefering with, knocking out, or mutating regulatory sequences (e.g., promoter, enhancer) associated with the gene).

Inhibition also includes inhibition of the gene product (e.g., protein, peptide), e.g., preventing the use by the plant of the gene's expression product, by mutating the gene product so that the gene product no longer retains its biological function or its biological function is significantly diminished; mutating a molecule with which the gene product interacts; introducing a molecule (e.g., peptide, small molecule) that binds the gene product thereby inhibiting its function. Inhibition also includes inhibiting a gene or gene product upstream in the pathway in which the particular gene is involved, e.g., a gene in the BON1 pathway which, when inhibited, results in inhibition of BON1 downstream.

One method of inhibiting expression of an endogenous gene is cosuppression. This has been shown in petunia where introduction of a recombinant chalcone synthase or dihydroflavonol4-reductase gene suppressed the homologous native genes (Napoli, C. et al., 1990, The Plant Cell 2:279-289; van der Krol, A. R. et al., 1990, The Plant Cell 2:291-299), and in tobacco, where transformation of a partial nopaline synthase gene into the plant suppresses the expression of the endogenous corresponding gene (Goring, D. R. et al., 1991, Proc. Natl. Acad. Sci. USA 88:1770-1774). In a particular embodiment, expression of a truncated form of the relevant gene in the “sense” orientation can be used to suppress the endogenous expression of the native gene, thus lowering the level of the gene product.

Another method of inhibiting expression of a gene is supression or inhibition using antisense techniques. In one embodiment, antisense RNA forms double-stranded RNA with a target gene product, thereby inhibiting action by that gene product. This is generally done by linking, in reverse orientation, the nucleic acid encoding the target gene product, downstream of its promoter, into a vector. The nucleic acid encoding the antisense product need not represent the entire gene, nor does it need to be fully homologous to the target RNA, rather, the antisense nucleic acid need only encode an RNA having enough homology to bind to the target RNA, or to be of sufficient length to bind to the target RNA in a region critical to its activity. One could use an antisense construct against the BON1, BON2 and/or BON3 proteins, for instance, to produce plants exhibiting the bon1 loss-of-function phenotype.

In a particular embodiment of the invention, a transgenic plant that is smaller in size than a corresponding wild type plant is produced by introducing a chimeric fusion protein, such as a BON1-GUS fusion protein (or a BON2-GUS or BON3-GUS fusion protein) into the plant (e.g., into the second exon or the twelfth exon of BON1) inhibits BON1 (or BON2 or BON3). In another embodiment, introduction of exogenous nucleic acid which overexpresses of the C-terminus of BON1, BON2 or BON3, or of an N-terminal domain of BAP1 can also be used to inhibit the genes.

In another embodiment, the present invention relates to a transgenic plant that is larger in size than a corresponding non-transgenic plant, wherein the transgenic plant comprises an exogenous nucleic acid which enhances BON1, BON2, BON3 and/or BAP1 in the plant.

The enhancing of BON1, BON2, BON3 or BAP1 can be partial or complete. Because BON1, BON2, BON3 and BAP1 are likely involved in a physiological pathway, “enhancing” of BON1, BON2, BON3 or BAP1 can include enhancing the gene, including duplicating and/or overexpressing the gene, mutating the gene (full length) in a way that it has increased expression, etc. Enhancing also includes interfering with the regulation of the gene, e.g., enhancing its expression or the timing thereof, e.g., altering regulatory sequences associated with the gene. Enhancing also includes enhancing the gene product itself, e.g., increasing the ability of the plant to use the product or increasing the activity of the product, either by mutating the gene product so that its use by the plant is increased, or by increasing binding of the expression product to its ligand.

Polynucleotides encoding BON1, BON2, BON3, BAP1 or BAL can be obtained or isolated from natural sources, recombinantly produced, or chemically synthesized. In one embodiment, polynucleotides encoding BON1, BON2, BON3, BAP1 or BAL can be cloned out of isolated DNA or a cDNA library. Nucleic acids and polypeptides, referred to herein as “isolated” (e.g., essentially pure) are nucleic acids or polypeptides substantially free (i.e., separated away from) the material of the biological source from which they were obtained (e.g., as exists in a mixture of nucleic acids or in cells), and which may have undergone further processing. An isolated nucleic acid is not immediately contiguous with (i.e., covalently linked to) both of the nucleic acids with which it is immediately contiguous in the naturally-occurring genome of the organism from which the nucleic acid is derived.

As used herein, “lower temperature” refers to a temperature at which the transgenic plant of the present invention grows smaller in size, compared to a corresponding non-transgenic plant. Such a lower temperature will vary depending upon the particular plant. For example, the normal temperature for a plant which grows in a temperate region (e.g., Arabidopsis) is from about 24° C. ro about 37° C. Transgenic Arabidopsis in which BON1, BON2, BON3, BAP1 and/or BAL are inhibited grows smaller in size at about 22° C., compared to a corresponding non-transgenic Arabidopsis plant at 22° C. Therefore, a lower temperature for plants which grow in a temperate region is from about 0° C. to about 23° C., from about 5° C. to about 20° C., and from about 10° C. to about 15° C. In one embodiment, a lower temperature for a plant is about 22° C. Thus, a “lower temperature” will vary depending upon the particular plant and the region (e.g., tropical, temperate, arctic) in which the wild type plant grown and can be determined by one of ordinary skill in the art.

Plants that are “smaller in size” are smaller than the corresponding wild type plant, and refers to their growth during the time of exposure to lower temperatures relative to wild type plants. For instance, as described herein, bon1 mutant plants do not grow as large, and produce smaller and fewer cells, when grown at 22° C. relative to wild type plants grown at the same temperature. Ordinarily, plants grown at lower temperatures continue to grow at a slower rate, but still produce normal-sized cells. If the bon1 plants are grown at 22° C. are then moved to exposure to 28° C., however, the new growth of bon1 plants grow at the same rate as the wild type plants, with the bon1 plants having a portion of older tissue that is “minaturized”, and newer tissue that is normally proportioned. The same is true if the temperature conditions are reversed, that is, if the bon1 plants are grown at one temperature (e.g., 28° C.), then switched to lower temperatures (e.g., 22° C.), the bon1 plants exhibit older tissue of the same proportions as wild type, while the newer tissues are miniaturized.

Conversely, transgenic plants of the present invention that are larger in size are larger than the corresponding wild type plant.

The invention also encompasses alleles, degenerate variants, fragments, mutants, homologs and analogs of BON1, BON2, BON3, BAP1 and BAL nucleic acids and proteins. An “allele” of a protein is a polypeptide sequence containing a naturally-occurring sequence variation relative to the polypeptide sequence of the reference polypeptide. By “allele” of a polynucleotide encoding the polypeptide is meant a polynucleotide containing a sequence variation relative to the reference polynucleotide sequence encoding the reference polypeptide, where the allele of the polynucleotide encoding the polypeptide encodes an allelic form of the polypeptide.

As used herein, “degenerate variants” of a nucleic acid are those variant nucleic acids that differ from the reference sequence due to the degeneracy of the genetic code, and encode a protein having the same amino acid sequence as that protein encoded by the reference nucleic acid. Therefore, a degenerate variant of a BON1, BON2, BON3, BAP1 or BAL nucleic acid sequence is a sequence that contains a silent or conservative substitution and therefore encodes the same protein as the reference polynucleotide sequence.

A “fragment” of the nucleic acid sequences described herein is a portion of the full length nucleic acid sequence that encodes a protein that retains the biological activity of the full-length protein. Such fragments con comprises, for example, from about 50 to about 2000 nucleotides, from about 100 to about 1500 nucleotides, from about 500 to about 1000 nucleotides. Such nucleic acids can also comprise additional sequences derived from the process of cloning, e.g., amino acid residues or amino acid sequences corresponding to full or partial linker sequences. To be encompassed by the present invention, such fragments, with or without such additional sequences, must have substantially the same biological activity as the natural or full-length version of the reference polypeptide.

A “fragment” of a protein is any amino acid sequence shorter than the full length protein, comprising at least about 25 consecutive amino acids of the full length protein. Such a fragment may alternatively comprise about 26, 27, 28, 29, 30, 31, 32, 33, 34, 35, 36, 37, 38, 39, 40, 41, 42, 43, 44, 45, 46, 47, 48, 49 or 50 consecutive amino acids of the full length protein. The fragment may comprise about 51, 52, 53, 54, 55, 56, 57, 58, 59, 60, 61, 62, 63, 64, 65, 66, 67, 68, 69, 70, 71, 72, 73, 74 or 75 consecutive amino acids of the full polypeptide. The fragments of the proteins described herein can comprise, for example, from about 100 to about 800 amino acids, from about 200 to about 700 amino acids, from about 300 to about 600 amino acids, and from about 500 to about 500 amino acids. In another embodiment, the protein fragment can be about 170 consecutive amino acids. In another embodiment, the protein fragment can be about 180 consecutive amino acids. In another embodiment, the protein fragment can be about 315 consecutive amino acids. In another embodiment, the protein fragment can be about 390 consecutive amino acids. In another embodiment, the protein fragment can be about 400 consecutive amino acids. In another embodiment, the protein fragment can be about 550 consecutive amino acids. Such molecules can comprise additional amino acids derived from the process of cloning, e.g., amino acid residues or amino acid sequences corresponding to full or partial linker sequences. To be encompassed by the present invention, such molecules, with or without such additional amino acid residues, must have substantially the same biological activity as the reference polypeptide.

Encompassed by the present invention are proteins that have substantially the same amino acid sequence as BON1, BON2, BON3, BAP1 or BAL, or polynucleotides that have substantially the same nucleic acid sequence as the polynucleotides encoding BON1, BON2, BON3, BAP1 or BAL. “Substantially the same sequence” means a nucleic acid or polypeptide that exhibits at least about 70% sequence identity, typically at least about 80% sequence identity with the reference sequence, at least about 90% sequence identity, at least about 95% identity, at least about 97%, at least about 98% sequence identity, or at least about 99% sequence identity with the BON1, BON2, BON3, BAP1 or BAL reference sequence. The length of comparison for sequences will generally be at least 75 nucleotide bases or 25 amino acids, more preferably at least 150 nucleotide bases or 50 amino acids, more preferably at least 300 nucleotides or 100 amino acids, and most preferably 600 nucleotides or 200 amino acids. In one embodiment, the length for comparisn is the full length nucleic acid or amino acid of BON1, BON2, BON3, BAP1 or BAL.

“Sequence identity,” as used herein, refers to the subunit sequence similarity between two polymeric molecules, e.g., two polynucleotides or two polypeptides. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length) of the positions in two peptide or compound sequences are identical, then the two sequences are 50% identical; if 90% of the positions, e.g., 9 of 10 are matched, the two sequences share 90% sequence identity. By way of example, the amino acid sequences R₂R₅R₇R₁₀R₆R₃ and R₉R₈R₁R₁₀R₆R₃ have 3 of 6 positions in common, and therefore share 50% sequence identity, while the sequences R₂R₅R₇R₁₀R₆R₃ and R₈R₁R₁₀R₆R₃ have 3 of 5 positions in common, and therefore share 60% sequence identity. The identity between two sequences is a direct function of the number of matching or identical positions. Thus, if a portion of the reference sequence is deleted in a particular peptide, that deleted section is not counted for purposes of calculating sequence identity, e.g., R₂R₅R₇R₁₀R₆R₃ and R₂R₅R₇R₁₀R₃ have 5 out of 6 positions in common, and therefore share 83.3% sequence identity.

Identity is often measured using sequence analysis software e.g., BLASTN or BLASTP (available at http://www.ncbi.nlm.nih.gov/BLAST/). The default parameters for comparing two sequences (e.g., “Blast”-ing two sequences against each other, http://www.ncbi.nlm.nih.gov/gorf/b12.html) by BLASTN (for nucleotide sequences) are reward for match=1, penalty for mismatch=−2, open gap=5, extension gap=2. When using BLASTP for protein sequences, the default parameters are reward for match=0, penalty for mismatch=0, open gap=11, and extension gap=1.

The present invention also includes fusion proteins and chimeric proteins comprising the BON1, BON2, BON3, BAP1 or BAL proteins, their fragments, mutants, homologs, analogs, and allelic variants. A fusion or chimeric protein can be produced as a result of recombinant expression and the cloning process, e.g., the protein can be produced comprising additional amino acids or amino acid sequences corresponding to full or partial linker sequences. As used herein, the term “fusion or chimeric protein” is intended to encompass changes of this type to the original protein sequence. A fusion or chimeric protein can consist of a multimer of a single protein, e.g., repeats of the proteins, or the fusion and chimeric proteins can be made up of several proteins, e.g., several of the proteins. Such fusion or chimeric proteins can be linked together via post-translational modification (e.g., chemically linked), or the entire fusion protein may be made recombinantly.

The invention also encompasses vectors and host cells comprising the BON1, BON2, BON3, BAP1 or BAL nucleic acid sequences. In addition, the present invention encompasses methods of producing BON1, BON2, BON3, BAP1 or BAL, and their fragments, mutants, homologs, analogs and allelic variants comprising culturing the host cells described herein under conditions appropriate to produce BON1, BON2, BON3, BAP1 and BAL. The term “vector” as used herein means a carrier into which pieces of nucleic acid may be inserted or cloned, which carrier functions to transfer the pieces of nucleic acid into a host cell. Such a vector may also bring about the replication and/or expression of the transferred nucleic acid pieces. Examples of vectors include nucleic acid molecules derived, e.g., from a plasmid, bacteriophage, or mammalian, plant or insect virus, or non-viral vectors such as ligand-nucleic acid conjugates, liposomes, or lipid-nucleic acid complexes. It may be desirable that the transferred nucleic molecule is operatively linked to an expression control sequence to form an expression vector capable of expressing the transferred nucleic acid. Such transfer of nucleic acids is generally called “transformation,” and refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Operably linked” refers to a situation wherein the components described are in a relationship permitting them to function in their intended manner, e.g., a control sequence “operably linked” to a coding sequence is ligated in such a manner that expression of the coding sequence is achieved under conditions compatible with the control sequence. A “coding sequence” is a polynucleotide sequence which is transcribed into mRNA and translated into a polypeptide when placed under the control of (e.g., operably linked to) appropriate regulatory sequences. The boundaries of the coding sequence are determined by a translation start codon at the 5′-terminus and a translation stop codon at the 3′-terminus. Such boundaries can be naturally-occurring, or can be introduced into or added the polynucleotide sequence by methods known in the art. A coding sequence can include, but is not limited to, mRNA, cDNA, and recombinant polynucleotide sequences.

Once a polynucleotide has been cloned into a suitable vector, it can be transformed into an appropriate host cell. By “host cell” is meant a cell which has been or can be used as the recipient of transferred nucleic acid by means of a vector. Host cells can be prokaryotic or eukaryotic, mammalian, plant, or insect cells, and can exist as single cells, or as a collection, e.g., as a culture, or in a tissue culture, or in a tissue or an organism. Host cells can also be derived from normal or diseased tissue from a multicellular organism, e.g., a plant. Host cell, as used herein, is intended to include not only the original cell which was transformed with a nucleic acid, but also descendants of such a cell, which still contain the nucleic acid.

In one embodiment, the isolated polynucleotide encoding one of the proteins of the present invention additionally comprises a polynucleotide linker encoding a peptide. Such linkers are known to those of skill in the art and, for example the linker can comprise at least one additional codon encoding at least one additional amino acid. Typically the linker comprises one to about twenty or thirty amino acids. The polynucleotide linker is translated, as is the polynucleotide encoding the protein, resulting in the expression of a protein with at least one additional amino acid residue at the amino or carboxyl terminus of the protein. Importantly, the additional amino acid, or amino acids, do not compromise the activity of the protein.

The present invention also relates to an isolated nucleic acid comprising a sequence that hybridizes under highly stringent conditions to a coding sequence of the isolated nucleic acid of BON1, BON2, BON3, BAP1 or BAL. The present invention also relates to an isolated nucleic acid sequence comprising a sequence that hybridizes under highly stringent conditions to a complement (e.g., fully complementary) of the coding sequence which encodes a BON1, BON2, BON3, BAP1 or BAL protein. The polynucleotides and proteins of the present invention can be used to design probes to isolate other proteins, for example, homologs of BON, BON2, BON3, BAP1 and BAL. Appropriate hybridization methods are provided in U.S. Pat. No. 5,837,490, by Jacobs et al., the entire teachings of which are herein incorporated by reference in their entirety. The design of the oligonucleotide probe should preferably follow these parameters: (a) it should be designed to an area of the sequence which has the fewest ambiguous bases (“N's”), if any, and (b) it should be designed to have a Tₘ of approx. 80° C. (assuming 2° C. for each A or T and 4° C. for each G or C).

The oligonucleotide should preferably be labeled with γ-³²P-ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4×10⁶ dpm/pmole. The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters, followed by lysing, denaturing and baking them. Highly stringent condition are those that are at least as stringent as, for example, 1× SSC at 65° C., or 1× SSC and 50% formamide at 42° C. Moderate stringency conditions are those that are at least as stringent as 4× SCC at 65° C., or 4× SCC and 50% formamide at 42° C. Reduced stringency conditions are those that are at least as stringent as 4× SCC at 50° C., or 6× SCC and 50% formamide at 40° C.

The filter is then preferably incubated at 65° C. for 1 hour with gentle agitation in 6× SCC (20× stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to 1×10⁶ dpm/mL. The filter is then preferably incubated at 65° C. with gentle agitation overnight. The filter is then preferably washed in 500 mL of 2× SCC/0.5% SDS at room temperature without agitation, preferably followed by 500 mL of 2× SCC/0.1% SDS at room temperature with gentle shaking for 15 minutes. A third wash with 0.1× SCC/0.5% SDS at 65° C. for 30 minutes to 1 hour is optional. The filter is then preferably dried and subjected to autoradiography for sufficient time to visualize the positives on the X-ray film. Other known hybridization methods can also be employed. The positive colonies are then picked, grown in culture, and plasmid DNA isolated using standard procedures. The clones can then be verified by restriction analysis, hybridization analysis, or DNA sequencing.

Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences may be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1× SSC (10× SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 1× SSC, 50% formamide, 1% SDS, 0.1-2 mg/ml denatured salmon sperm DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5× SCC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5× SSC, 5× Denhardt's solution, 1% SDS, 100 μg/ml denatured salmon sperm DNA at 65° C., or (6) 5× SCC, 5× Denhardt's solution, 50% formamide, 1% SDS, 100 μg/ml denatured salmon sperm DNA at 42° C., with high stringency washes of either (1) 0.3-0.1× SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4× SCC, (10× SCC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 4× SCC, 50% formamide, 1% SDS, 0.1-2 mg/ml denatured salmon sperm DNA at 42° C., (3) 1% bovine serum albumen (fraction V), 1 mM Na₂-EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5× SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5x SSC, 5× Denhardt's solution, 1% SDS, 100 μg/ml denatured salmon sperm DNA at 65° C., or (6) 5× SCC, 5× Denhardt's solution, 50% formamide, 1% SDS, 100 μg/ml denatured salmon sperm DNA at 42° C., with moderate stringency washes of 1× SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4×the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Low stringency conditions can employ hybridization at either (1) 4× SCC, (10× SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 50° C., (2) 6× SCC, 50% formamide, 1% SDS, 0.1-2 mg/ml denatured salmon sperm DNA at 40° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 50° C., (4) 50% formamide, 5× SCC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 40° C., (5) 5× SSC, 5× Denhardt's solution, 1% SDS, 100 μg/ml denatured salmon sperm DNA at 50° C., or (6) 5× SCC, 5× Denhardt's solution, 50% formamide, 1% SDS, 100 μg/ml denatured salmon sperm DNA at 40° C., with low stringency washes of either 2× SCC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2×the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

The present invention also relates to a method of producing a transgenic plant, wherein the size of the plant is altered in comparison to the size of a corresponding wild type plant, comprising introducing into the plant an exogenous nucleic acid that modulates (inhibits, enhances) BON1, BON2, BON3, BAP1, and/or BAL. The invention also relates to plants produced by the methods. In one embodiment, the present invention relates to a method of producing a transgenic plant that is smaller in size than a corresponding wild type plant, comprising introducing into the plant an exogenous nucleic acid which inhibits BON1, BON2, BON3, BAP1 and/or BAL in the plant. In another embodiment, the present invention relates to a method of producing a transgenic plant that is larger in size than a corresponding non-transgenic plant, comprising introducing into the plant an isolated nucleic acid which enhances expression of BON1, BON2, BON3, BAP1 and/or BAL protein in the plant.

The nucleic acids of the present invention can be used in a variety of ways. In one embodiment, the present invention relates to methods of producing plants which are miniature in size, comprising introducing exogenous nucleic acid which inhibits BON1, BON2, BON3, BAP1, or BAL in the plant. In another embodiment, the present invention relates to methods of increasing the yield of a plant, comprising introducing into the plant an isolated nucleic acid which enhances BON1, BON2, BON3, BAP1, or BAL in the plant. In another embodiment, the present invention relates to methods of producing a transgenic plant that is able to grow at a higher altitude or in a lower temperature region than a corresponding non-transgenic plant, comprising introducing into the plant an isolated nucleic acid that enhances BON1, BON2, BON3, BAP1, or BAL in the plant. In another embodiment, the present invention relates to a method of modulating homeostasis of a plant, the method comprising introducing into the plant an exogenous nucleic acid which modulates BON1 in the plant. The present invention also relates to the plants produced by such methods, where the plants are miniature in size, are higher-yielding, grow at higher altitues or in cooler regions, or in which homeostasis regarding temperature and plant growth are modulated by the nucleic acids of the invention.

To produce transgenic plants of this invention, a construct comprising exogenous nucleic acid of the invention, or nucleic acid encoding a functional equivalent as described herein, and a promoter can be incorporated into a vector and introduced into the cell(s) of the plant through methods known and used by those of skill in the art. The nucleic acid to be introduced can be incorporated into a vector, to form a construct, with or without other sequences, or it may be naked nucleic acid, and associated with no such sequences. The construct can also include any other necessary regulators such as terminators or the like, operably linked to the coding sequence. It can also be beneficial to include a 5′ leader sequence, such as the untranslated leader from the coat protein mRNA of alfalfa mosaic virus (Jobling, S. A. and Gehrke, L. (1987) Nature 325:622-625) or the maize chlorotic mottle virus (MCMV) leader (Lommel, S. A. et al. (1991) Virology 81:382-385). Those of skill in the art will recognize the applicability of other leader sequences for various purposes.

Targeting sequences are also useful and can be incorporated into the constructs of this invention. A targeting sequence is usually translated into a peptide which directs the polypeptide product of the coding nucleic acid sequence to a desired location within the cell, such as to the plastid, and becomes separated from the peptide after transit of the peptide is complete or concurrently with transit. Examples of targeting sequences useful in this invention include, but are not limited to, the yeast mitochondrial presequence (Schmitz et al. (1989) Plant Cell 1:783-791), the targeting sequence from the pathogenesis-related gene (PR-1) of tobacco (Comellisen et al. (1986) EMBO J 5:37-40), vacuole targeting signals (Chrispeels, M. J. and Raikhel, N. V. (1992) Cell 68:613-616), secretory pathway sequences such as those of the ER or Golgi (Chrispeels, M. J. (1991) Ann. Rev. Plant Physiol. Plant Mol. Biol. 42:21-53). Intraorganellar sequences may also be useful for internal sites, e.g., thylakoids in chloroplasts. Theg, S. M. and Scott, S. V. (1993) Trends in Cell Biol. 3:186-190.

In addition to 5′ leader sequences, terminator sequences are usually incorporated into the construct. In plant constructs, a 3′ untranslated region (3′ UTR) is generally part of the expression plasmid and contains a polyA termination sequence. The termination region which is employed will generally be one of convenience, since termination regions appear to be relatively interchangeable. The octopine synthase and nopaline synthase termination regions, derived from the Ti-plasmid of A. tumefaciens, are suitable for such use in the constructs of this invention.

The transcriptional initiation region of the construct may provide for constitutive expression or regulated expression. In addition to the ERA1 promoter, many promoters are available which are functional in plants.

Constitutive promoters for plant gene expression include, but are not limited to, the octopine synthase, nopaline synthase, or mannopine synthase promoters from Agrobacterium, the cauliflower mosaic virus (35S) promoter, the figwort mosaic virus (FMV) promoter, and the tobacco mosaic virus (TMV) promoter. Constitutive gene expression in plants can also be provided by the glutamine synthase promoter (Edwards et al. (1990) Proc. Natl. Acad. Sci. USA 87:3459-3463), the maize sucrose synthetase 1 promoter (Yang et al. (1990) Proc. Natl. Acad. Sci. USA 87:4144-4148), the promoter from the Rol-C gene of the TLDNA of Ri plasmid (Sagaya et al. (1989) Plant Cell Physiol. 30:649-654), and the phloem-specific region of the pRVC-S-3A promoter (Aoyagi et al. (1988) Mol. Gen. Genet. 213:179-185).

Heat-shock promoters, the ribulose-1,6-bisphosphate (RUBP) carboxylase small subunit (ssu) promoter, tissue specific promoters, and the like can be used for regulated expression of plant genes. Developmentally-regulated, stress-induced, wound-induced or pathogen-induced promoters are also useful.

The regulatory region may be responsive to a physical stimulus, such as light, as with the RUBP carboxylase ssu promoter, differentiation signals, or metabolites. The time and level of expression of the sense or antisense orientation can have a definite effect on the phenotype produced. Therefore, the promoters chosen, coupled with the orientation of the exogenous DNA, and site of integration of a vector in the genome, will determine the effect of the introduced gene.

Specific examples of regulated promoters also include, but are not limited to, the low temperature Kin1 and cor6.6 promoters (Wang et al (1995) Plant Mol. Biol. 28:605; Wang et al. (1995) Plant Mol. Biol. 28:619-634), the ABA inducible promoter (Marcotte Jr. et al. (1989) Plant Cell 1:969-976), heat shock promoters, such as the inducible hsp70 heat shock promoter of Drosphilia melanogaster (Freeling, M. et al. (1985) Ann. Rev. of Genetics 19: 297-323), the cold inducible promoter from B. napus (White, T. C. et al. (1994) Plant Physiol. 106:917), the alcohol dehydrogenase promoter which is induced by ethanol (Nagao, R. T. et al., Miflin, B. J., Ed. Oxford Surveys of Plant Molecular and Cell Biology, Vol. 3, p 384-438, Oxford University Press, Oxford 1986), the phloem-specific sucrose synthase ASUS1 promoter from Arabidopsis (Martin et al. (1993) Plant J. 4:367-377), the ACS1 promoter (Rodrigues-Pousada et al. (1993) Plant Cell 5:897-911), the 22 kDa zein protein promoter from maize (Unger et al. (1993) Plant Cell 5:831-841), the ps1 lectin promoter of pea (de Pater et al. (1993) Plant Cell 5:877-886), the phas promoter from Phaseolus vulgaris (Frisch et al. (1995) Plant J. 7:503-512), the lea promoter (Thomas, T. L. (1993) Plant Cell 5:1401-1410), the E8 gene promoter from tomato (Cordes et al. (1989) Plant Cell 1:1025-1034), the PCNA promoter (Kosugi et al. (1995) Plant J. 7:877-886), the NTP303 promoter (Weterings et al. (1995) Plant J. 8:55-63), the OSEM promoter (Hattori et al. (1995) Plant J. 7:913-925), the ADP GP promoter from potato (Muller-Rober et al. (1994) Plant Cell 6:601-604), the Myb promoter from barley (Wissenbach et al. (1993) Plant J. 4:411-422), and the plastocyanin promoter from Arabidopsis (Vorst et al. (1993) Plant J. 4:933-945).

The nucleic acid can be introduced into plant cells by a method appropriate to the type of host cells (e.g., transformation, electroporation, transfection, infection). For the purposes of this disclosure, the terms “transformed with”, “transformant”, “transformation”, “transfect with”, and “transfection” all refer to the introduction of a nucleic acid into a cell by one of the numerous methods known to persons skilled in the art. Transformation of prokaryotic cells, for example, is commonly achieved by treating the cells with calcium chloride so as to render them “competent” to take up exogenous DNA, and then mixing such DNA with the competent cells. Prokaryotic cells can also be infected with a recombinant bacteriophage vector.

Any suitable technique can be used to introduce the nucleic acids and constructs of this invention to produce transgenic plants with an altered genome. Nucleic acids can be introduced into cells of higher organisms by viral infection, bacteria-mediated transfer (e.g., Agrobacterium T-DNA delivery system), electroporation, calcium phosphate co-precipitation, microinjection, lipofection, bombardment with nucleic-acid coated particles, mixing with silicon carbide “whiskers”, floral dip method or other techniques, depending on the particular cell type. “Introduction”, as used herein, of a nucleic acid into a plant, plant cell, plant part or tissue culture, is intended to include both those methods of transformation that are known and used with single-celled organisms (e.g., bacteria, yeast, etc.), and also those methods that are known and used for moving nucleic acid into plants, plant cells, plant parts and tissue cultures.

For grasses such as maize and sorghum, for instance, microprojectile bombardment (see for example, Sanford, J. C. et al., U.S. Pat. No. 5,100,792 (1992) can be used. In this embodiment, a nucleotide construct or a vector containing the construct is coated onto small particles which are then introduced into the targeted tissue (cells) via high velocity ballistic penetration. The vector can be any vector which permits the expression of the exogenous DNA in plant cells into which the vector is introduced. The transformed cells are then cultivated under conditions appropriate for the regeneration of plants, resulting in production of transgenic plants.

Other useful protocols for the transformation of plant cells are provided in Gelvin et al., 1992. Suitable protocols for transforming and transfecting cells are also found in Sambrook et al., 1989. The nucleic acid constructs of this invention can also be incorporated into specific plant parts through the transformation and transfection techniques described herein. The constructs and methods of this invention can be adapted to any plant part, protoplast, or tissue culture wherein the tissue is derived from a photosynthetic organism. The term “plant part” is meant to include a portion of a plant capable of producing a regenerated plant. Preferable plant parts include cells, roots, shoots and meristematic portions thereof. Other plant parts encompassed by this invention are: leaves, stems, roots, flowers, seeds, epicotyls, hypocotyls, cotyledons, cotyledonary nodes, explants, pollen, ovules, meristematic or embryonic tissue, protoplasts, cells, explants and the like. Transgenic plants can be regenerated from any of these plant parts, including tissue culture or protoplasts, and also from explants. Methods will vary according to the species of plant.

Other known methods of inserting nucleic acid constructs into plants include Agrobacterium-mediated transformation (see for example Smith, R. H. et al., U.S. Pat. No. 5,164,310 (1992)), electroporation (see for example, Calvin, N., U.S. Pat. No. 5,098,843 (1992)), introduction using laser beams (see for example, Kasuya, T. et al., U.S. Pat. No. 5,013,660 (1991)) or introduction using agents such as polyethylene glycol (see for example Golds, T. et al. (1993) Biotechnology 11:95-97), and the like. In general, plant cells may be transformed with a variety of vectors, such as viral, episomal vectors, Ti plasmid vectors and the like, in accordance with well known procedures. The method of introduction of the nucleic acid into the plant cell is not critical to this invention.

To aid in identification of transformed plant cells, the constructs of this invention are further manipulated to include genes coding for plant selectable markers. Useful selectable markers include enzymes which provide for resistance to an antibiotic such as gentamycin, hygromycin, kanamycin, or the like. Similarly, enzymes providing for production of a compound identifiable by color change such as GUS (β-glucuronidase), or by luminescence, such as luciferase, are useful.

When cells or protoplasts containing the nucleic acids of the invention are obtained, the cells or protoplasts are regenerated into whole plants. Plant cells which have been transformed can be regenerated using known techniques. It is known that practically all plants can be regenerated from cultured cells or tissues. The transformed cells are then cultivated under conditions appropriate for the regeneration of plants, resulting in production of transgenic plants. Choice of methodology for the regeneration step is not critical, with suitable protocols being available for many varieties of plants, tissues and other photosynthetic organisms. See, e.g., Gelvin S. B. and Schilperoort R. A., eds. Plant Molecular Biology Manual, Second Edition, Suppl. 1 (1995) Kluwer Academic Publishers, Boston Mass., USA. Plant regeneration from cultured protoplasts is also described in Evans et al., Handbook of Plant Cell Cultures, Vol. 1, MacMillan Publishing Co. New York, 1983; and Vasil I. R., ed., Cell Culture and Somatic Cell Genetics of Plants, Academic Press, Orlando, Fla., USA, Vol. 1, 1984, and Vol. II, 1986).

Means for regeneration vary from species to species of plants, but generally a suspension of transformed protoplasts or a petri plate containing transformed explants is first provided. Callus tissue is formed and shoots may be induced from callus and subsequently rooted. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable. The regenerated plants are transferred to standard soil conditions and cultivated in a conventional manner.

Because of the plasticity of culture of many plant species, the terms “plant”, “plant part”, “plant cell”, and “tissue culture” are intended to be used somewhat interchangably herein. For instance, it is possible, indeed common in the art of plant transformation and tissue culture, for one to transform either a plant, a plant part, one or more plant cells, and/or a tissue culture. Once transformed, any of these may be maintained for a time, converted to another, subcultured, and regenerated into a whole plant, from which seed may then be grown. As an example, one can use microparticle bombardment to transform cells in a single leaf on a plant. The plant may then be grown for a time (or not), and the leaf then separated from the plant, and either maintained on growth medium as a leaf, or (through changing the media components) grown as callus (undifferentiated tissue) or protoplasts (single plant cells). At such point, the callus or protoplasts may be either subcultured further as callus or protoplasts, or the callus may be converted to protoplasts, and vice versa. Either may be grown to generate individual plant parts (by manipulation of the hormone conditions in the media to preferentially produce roots and/or shoots, etc.), or to regenerate into whole plants (also by hormone manipulation). The regenerated plant may then again be cultured into plant parts, cuttings, plant cells, callus, tissue cultures, etc., or may be grown for seed. Because of this plasticity, “transgenic plant” is intended to encompass any subculturing, regenerant, seed, or progeny of the transgenic plant, that carries the introduced nucleic acid. It is also due to this plasticity that the term “derived from” is intended to encompass plants, plant parts, plant cells, tissue cultures and explants that are descended from a given plant. For instance, in the case where a descendant corn plant that has been regenerated from a callus culture which was grown from transformed protoplasts taken from a leaf of an originating maize plant, the descendant plant is said to be “derived from” the originating maize plant. The intervening protoplasts and callus are also said to be “derived from” the originating maize plant.

The methods of this invention can be used with in planta or seed transformation techniques which do not require culture or regeneration. Examples of these techniques are described in Bechtold, N. et al. (1993) C.R. Acad. Sci. Paris/Life Sciences 316:118-93; Chang, S. S. et al. (1990) Abstracts of the Fourth International Conference on Arabidopsis Research, Vienna, p. 28; Feldmann, K. A. and Marks, D. M (1987) Mol. Gen. Genet. 208:1-9; Ledoux, L. et al. (1985) Arabidopsis Inf. Serv. 22: 1-11; Feldmann, K. A. (1992) In: Methods in Arabidopsis Research (Eds. Koncz, C., Chua, N-H, Schell, J.) pp. 274-289; Chee et al., U.S. Pat. No. 5,376,543.

After the nucleic acid to be introduced is stably incorporated into regenerated transgenic plants, it can be transferred to other plants by sexual crossing. Any of a number of standard breeding techniques can be used, depending upon the species to be crossed. The plants are grown and harvested using conventional procedures. Such plants, if they continue to contain the introduced nucleic acid of the invention, are intended to be a “transgenic plant”, as the term is used herein.

Transgenic plants carrying the construct are examined for the desired phenotype using a variety of methods including, but not limited to, an appropriate phenotypic marker, such as antibiotic resistance or herbicide resistance, or visual observation, or biochemical or other methods of assaying the transgenic plant compared to a corresponding non-transgenic plant. By “corresponding non-transgenic plant” is meant a plant of the same species, and if applicable, same cultivar, variety, or genetic background, that was not transformed.

Any suitable plant can be used in the compositions and methods of the present invention. Such transgenic plants include, in one embodiment, transgenic plants which are angiosperms, both monocotyledons and dicotyledons. Transgenic plants include those into which DNA has been introduced and their progeny, produced from seed, vegetative propagation, cell, tissue or protoplast culture, or the like.

The constructs and methods described herein can be applied to all types of plants and other photosynthetic organisms, including, but not limited to: angiosperms (monocots and dicots), gymnosperms, spore-bearing or vegetatively-reproducing plants and the algae, including the cyanophyta (blue-green algae). Particularly preferred plants are those plants which provide commercially-valuable food crops such as the large and small grains (e.g., corn, wheat, rice, sorghum, etc.), oil plants (e.g., canola, sunflower, corn, soybean, peanut, etc.), vegetable crops (e.g., luttuce, spinach, pepper, potatoes, tomatoes, broccoli, carrots, peas, beans, etc.), fruits (e.g., apple, plum, orange, lemon, etc.), ornamental plants (e.g., rose, cut flowers, etc.), and other commercially-important plants (e.g., cotton, sugar cane, sugar beet, tobacco, grasses, etc.).

Seed can be obtained from the regenerated plant or from a cross between the regenerated plant and a suitable plant of the same species. Seed may also be obtained from plant parts, e.g., meristems grown in tissue culture. Alternatively, the plant can be vegetatively propagated by culturing plant parts under conditions suitable for the regeneration of such plant parts.

The nucleic acid and amino acid sequences of the present invention can be used in a variety of ways, e.g., the transgenic plants containing the BON1, BON2, BON3, the BAP1 and/or the BAL mutant(s) can be used to produce plants which grow slowly at lower temperatures. Such a trait would be useful in turf grasses, for instance, where it may be undesirable or labor-intensive to maintain, e.g., in low-traffic areas where the appearance of lawn is to be maintained, but the labor of mowing can be reduced.

Such miniaturized plants can be used in situations where a normal-sized plant or plant product is less desirable, e.g., miniaturized ornamental plants, such as smaller flower, vegetable or fruit plants for use by apartment residents, “no-care” bonsai trees, miniature water lilies, miniature fruits as novelties, etc., or low growth where extensive growth is undesirable.

Another embodiment of the invention is the production of “baby vegetables.” “Baby corn,” for instance, is very popular in Asian cuisine, and is the ear or maize which has been harvested young, before pollination has taken place, either before the silks have emerged, or just after, depending on the cultivar. Because baby corn is the immature ear of conventional maize, a great deal of land must be used to produce this vegetable, and the plants themselves are close to full height when the ears are produced.

By transforming a BON1-GUS fusion into maize protoplasts, as described below, bon1 corn plants can be made which grow as miniatures at cool temperatures. By sowing such plants in the fall in a relatively southern climate, e.g., southern Georgia or northern Florida, the plants will grow as miniatures during the cooler (but not freezing) winter. Such corn, planted for baby corn production, can be grown at a much higher density than standard-sized corn plants, and during a time of year when other crops are not grown. Other “baby” vegetables can also be grown in this way, such as baby turnips, eggplants, and carrots.

Another embodiment of the invention is a method of producing a transgenic plant that is able to grown at a higher altitude or in a cooler temperature region that a corresponding non-transgenic plant, comprising introducing into the plant an isolated nucleic acid that enhances BON1, BON2, BON3, BAP1 and/or BAL in the plant. The invention also encompases the plant produced by the method, where the plant are able to be grown at a higher altitude or in a cooler temperature region that a corresponding non-transgenic plant.

Another embodiment of the invention is the production of larger plants, or plants that grow at lower than normal temperatures or to modulate homeostasis at higher altitudes. Such plants can be produced by enhancing BON1, BON2, BON3 or BAP1, e.g., by overexpression as described herein. Such larger plants can be used in situations where larger fruits or vegetables are desired, or where increased biomass (total amount of vegetable matter produced by the plant per unit of land) is desired, e.g., for biomass for paper production.

### EXAMPLES

### Example 1

### Plant Growth Homeostasis is Controlled by the Arabidopsis BON1 and BAP1 Genes

Experimental Procedures

Isolation and Characterization of the bon1 Mutant

Several mutant pools from Arabidopsis Biological Resource Center were screened for leaf expansion mutants. Approximately 300 plants were grown per flat (26 cm×52 cm) for phenotypic screening. bon1-1 was isolated from the activation tagging lines in Col background (Weigel, D. et al. (2000) Plant Physiol. 122(4):1003-13).

For scanning microscopy, plant material was fixed overnight at 4° C. in 3% glutaraldehyde in 25 mM phosphate buffer (pH 7.0), dehydrated through ethanol serials, critical-point dried, gold sputter-coated, and examined by scanning electron microscope. Images were taken and the cell dimensions were analyzed using the NIH imaging program.

Cloning of the BON1 Gene

A 0.5 kb genomic fragment outside the left border of the T-DNA was rescued by using Universal Genome Walker kit (Clontech, Palo Alto, Calif., USA). The sequence of this 0.5 kb fragment revealed its location on TAC clone K22G18 from the Arabidopsis Biological Resource Center. A 6.5 kb BamHI fragment containing the gene was subcloned from this TAC clone. Using the same 0.5 kb fragment, a cDNA clone was isolated from an Arabidopsis cDNA library CD4-14 obtained from the Arabidopsis Biological Resource Center.

Constructs

For complementation, a 6.5 kb BamHI genomic fragment of BON1 was cloned into pCGN1548 (McBride, K. E. and K. R. Summerfelt (1990) Plant Mol. Biol. 14:269-276) and pCGN-NOS with 3′-NOS added in pCGN1548. For BON1-GFP fusion, a full-length cDNA of this gene was cloned into a GFP expression vector (Chiu, W., Y. Niwa et al. (1996) Curr. Biol. 6(3): 325-30). For BON1-GUS fusion, a 5 kb BamHI/BglII fragment was cloned into PZP212 vector (Diener, A. C., H. Li et al. (2000) Plant Cell 12(6):853-70). For BON1-HA fusion, a BamHI site was introduced by PCR method to the genomic BON1 gene to replace the stop codon. The 3×HA epitope was amplified and ligated into the BamHI site at the last codon of BON1 so that it would be in frame with BON1. The BON1-HA fusion was then inserted into pCGN-NOS.

RNA and DNA Analysis

Standard molecular techniques were used (Sambrook, J., E. F. Fritsch et al. (1989) Molecular Cloning, a Laboratory Manual, Cold Spring Harbor Laboratory Press, Cold Spring Harbor, N.Y., USA). Total RNA was prepared using Tri Reagent (Molecular Research Center, Inc., Cincinnati, Ohio, USA).

Plant Transformation

Plasmids were introduced into Agrobacterium strain ASE (Fraley, R. T., S. G. Rogers et al. (1985) Bio/Technology 3:629-635) or GV3101 (Koncz, C., J. Schell (1986) Mol. Gen. Genet. 204:383-396) by electroporation, and transformed into wild-type Col-0, bon1-1/+ or bon1-1 by the floral dip method (Clough, S. J. and A. F. Bent (1998) Plant J. 16(6):735-43).

GUS Staining

Plant material was prefixed on ice in 90% acetone for 15 minutes, rinsed in staining buffer without X-Gluc (cyclohexylammonium salt), infiltrated with staining solution (50 mM NaPO₄ pH 7.2, 0.5 mM K₃Fe(CN)₆, 0.5 mM K₄Fe(CN)₆, 2 mM XGluc) under vacuum for 15 minutes, and incubate at 37° C. for overnight. Materials were then dehydrated in an ethanol series.

Protein Analysis

For total protein extraction, rosettes were homogenized in extraction buffer (12% sucrose, 100 mM Tris-HCl pH 7.5, 1 mM EDTA, 2 mM DTT, and proteinase inhibitors (Calbiochem, La Jolla, Calif., USA) with chilled mortar and pestle. The mixture was spun at 15,000 rpm in a microcentrifuge for 5 minutes to remove insoluble debris. To assay the effects of different reagents on the association of BON1 with the membrane, supernatant was incubated for 1 hour on ice with equal volume of buffer to make the final concentration of 0.5 M NaCl, 0.1 M Na₂CO₃, 2.5 M urea, 1% TritonX100, and 1% sarcosyl, respectively. The soluble and pellet fractions were separated by centrifuging the supernatant at 100,000 g for 1 hour.

Western Blot

Proteins were run on 4-20% Ready-gel (Bio-Rad, Hercules, Calif., USA) and transferred to PVDF membrane (NEN Life Sciences, Inc. Products, Boston, Mass., USA). Membranes were blocked with 5% non-fat dry milk in PBT (phosphate-buffered saline with 0.1% Tween 20), incubated with primary and then secondary antibodies in 2% milk, and detected with ECL (Amersham Pharmacia Biotech, Arlington Heights, Ill., USA). HA antibody was purchased from BAbCo (Berkeley Antibody Company, Inc., Richmond, Calif., USA).

Membrane Fractionation and Analysis

The procedure was adapted from a method described previously (Schaller, G. E. and N. D. DeWitt (1995) Methods Cell Biol. 50:129-48). Briefly, 3-week-old leaves grown in liquid medium of MS salt, 2% sucrose, and B5 vitamins were homogenized in extraction buffer (50 mM Tris pH 8, 2 mM EDTA, 20% glycerol, 1 mM DTT, and the proteinase inhibitor cocktail (Calbiochem, La Jolla, Calif., USA). Homogenate was filtered through Miracloth (Calbiochem, La Jolla, Calif., USA) and centrifuged at 5,000g for 5 minutes to remove organelles and debris. Supernatants were centrifuged at 100,000 g for 1 hour to pellet microsomal membranes. Membrane pellets were resuspended in buffer (25 mM Tris pH 7.5, 10% sucrose, 2 mM EDTA, 1 mM DTT, and proteinase inhibitors) at 0.625 ml/10 g starting material. Microsomes (0.5 ml) were then layered onto a 15 ml sucrose gradient of 25% to 50% and centrifuged at 100,000 g for 16 hours. Fractions of 1 ml were collected and analyzed. Fractions containing ER, vacuole, and PM were identified by Western blot using marker enzymes. Fractions containing Golgi were identified by enyzmatic analysis of Triton-stimulated UDPase.

Arabidopsis Protoplast Transient Expression and Confocal Microscopy

Leaf mesophyll protoplasts were isolated from 3-week-old Arabidopsis plants and were transfected by a modified polyethylene glycol method as described previously (Abel, S. and A. Theologis (1994) Plant J. 5(3):421-7). 10,000 protoplasts were transfected with 10 μg of DNA and then incubated at 23° C. for 9 hours before subcelluar localization was determined with a confocal microscope. Confocal laser scanning micrographs of transfected protoplasts were recorded using a Leica microscope (Leica Microsystems, Heidelburg, Germany) equipped with a laser scanning unit (TCS NT).

Protein Expression in E. coli

Full length, 2C2 domain (M1 to G308), and A domain (V319 to P578) were amplified from BON1 cDNA and cloned into a vector pKS L 1 (under T7 promoter, with 6× His tags at the N-terminus). The constructs were transformed into BL21plyS (Novagen, Madison, Wis., USA). Protein was induced with 1 mM IPTG at 25° C. for 10 hours. Proteins were purified with Ni-NTA agarose beads (Qiagen, Valencia, Calif., USA) according to instructions.

Two Hybrid Screen

DNA was prepared from the Arabidopsis thaliana MATCHMAKER cDNA library in E. coli (Clontech, Palo Alto, Calif., USA). The library complexity was 3×10⁶. DNA was transformed into yeast strain PJ69-4α (James, P., J. Halladay et al. (1996) Genetics 144(4):1425-36) and 2.5×10⁷ yeast transformants were pooled as the library. The bait (A domain of BON1) was cloned between EcoRI and BamHI sites of pGBD-C2 and transformed into PJ49-4a. The library a cells were mated with the bait A cells. A screening protocol (Robertson, L. S., H. C. Causton et al. (2000) Proc. Natl. Acad. Sci. USA 97(11):5984-8) was followed. Approximately 10⁸ diploid cells were plated onto SC Ade⁻ and His-medium supplemented with 40 mM of 3-AT. About 80 clones retested positively were chosen for plasmid rescue.

Results

BON1 is a Gene Required for Normal Plant Size at 22° C.

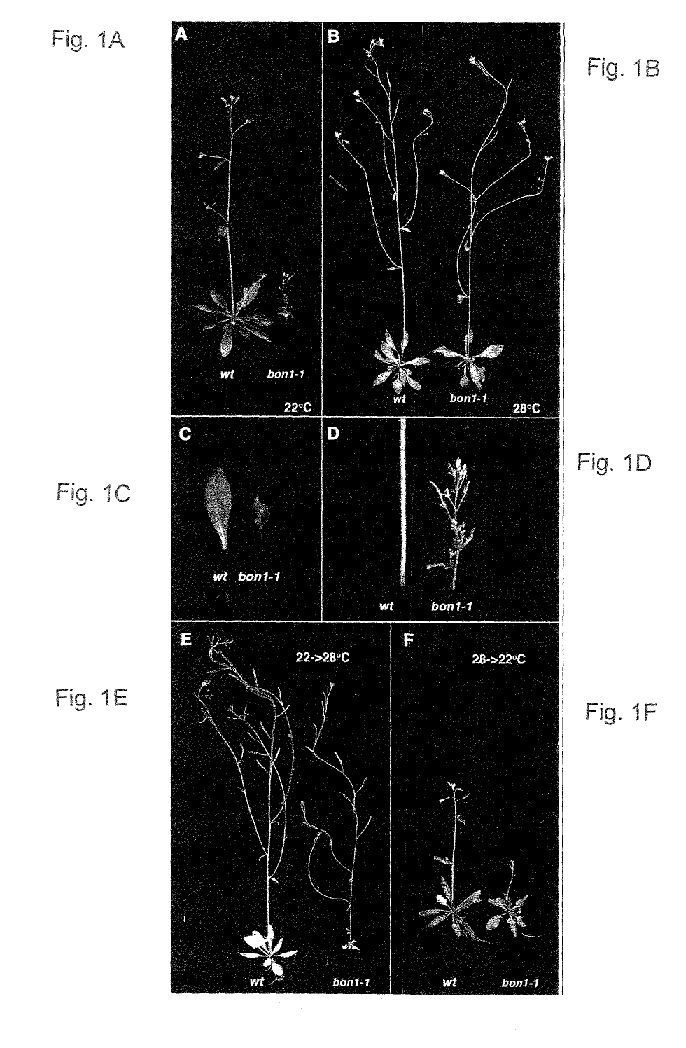

To obtain mutants that have a restricted temperature range for normal development, a screen was conducted for Arabidopsis strains that were miniature when grown at one temperature but not at another. This screen uncovered a mutant, bon1, that exhibits a dwarf phenotype at 22° C. (FIG. 1A), but has a phenotype indistinguishable from that of the wild type at 28° C. (FIG. 1B), which is the normal growth temperature for wild type Arabidopsis. This temperature dependent phenotype is not an allele-specific phenomenon because bon1-1 is a loss-of-function mutant (see below).

At 22° C., the bon1-1 leaves are greatly reduced in size and have a curved morphology (FIG. 1C). The inflorescence stem is thinner and shorter than the wild type (FIG. 1D). Despite the smaller size of the leaves and stems at 22° C., bon1-1 plants nevertheless develop relatively normal flowers and siliques and are completely fertile. bon1-1 does not have a dramatic effect on the timing of developmental process. It bolts and sets seeds only slightly earlier than the wild type. The bon1 phenotype is genetically distinct from that of previously identified hormonal dwarf mutants such as axr1, det2, or ctr1. The bon1-1 defect cannot be rescued by the addition of hormones such as gibberellin or brassinosteroid, or by mutations in hormonal signaling. Moreover, the bon1-1 mutant is distinct from the cold-sensitive mutants deficient in the desaturation of the lipids in the membranes (Lightner, J., D. W. J. James et al. (1994) Plant J. 6(3):401-412) because bon1-1 does not have an altered lipid composition or altered composition of saturated fatty acids in the lipids. These observations suggest that BON1 functions to maintain plant morphology through a novel mechanism.

BON1 is Required to Maintain Cell Size and Number at 22° C.

Using scanning electron microscope (SEM), the width and length of the epidermal cells of the inflorescence stems grown at 22° C. were measured. The results are shown in the Table below.

TABLE
Cell size and number of the stem epidermal cells in the wild type and bon1-1.

| | | | stem | | cell number | cell number |
|---|---|---|---|---|---|---|
| | stem length | cell length | diameter | cell width | per | per |
| | (cm) | (mm) | (cm) | (mm) | circumference | stem file |
| wild type | 18 | 288 ± 55 | 1.0 | 18± 2 | ˜174 | ˜625 |
| bon1-1 | 2.2 | 40 ± 9 | 0.38 | 12± 1 | ˜99 | ˜550 |

The length of the bon1-1 cells is approximately seven times shorter than that of the wild type, which accounts for most of the eight-fold reduction in bon1 stem length at the non-permissive temperature. A bon1-1 cell is about two-thirds as wide as the wild-type cell. There are also half as many cells per epidermal circumference in a bon1-1 mutant as there are in the wild type, although the number of cells along the length of the same was nearly the same as in the wild type. Analysis of whole stem cross sections revealed similar defects in the inner cells: there were fewer and smaller inner cells inside the stem. Reduction in the size of the pavement cells was observed by SEM on the leaf epidermis. Taken together, these data show that the small size of bon1-1 during growth at 22° C. results from a reduction in both cell size and cell number.

Temperature shift experiments were used to determine at what period of development BON1 function is required for growth. When bon1-1 plants were shifted from 22° C. to 28° C., the new stems and leaves that formed at 28° C. had normal expansion and elongation in contrast with the miniature stems and leaves formed when plants were grown 22° C. (FIG. 1E). Conversely, after bon1-1 was shifted from 28° C. to 22° C., the new leaves and stems that formed at 22° C. had a -miniature phenotype (FIG. 1F). These data show that BON1 is required continuously for normal growth of leaves and stems at 22° C.

BON1 is Expressed in Growing Tissues and the Expression is Modulated by Temperature.

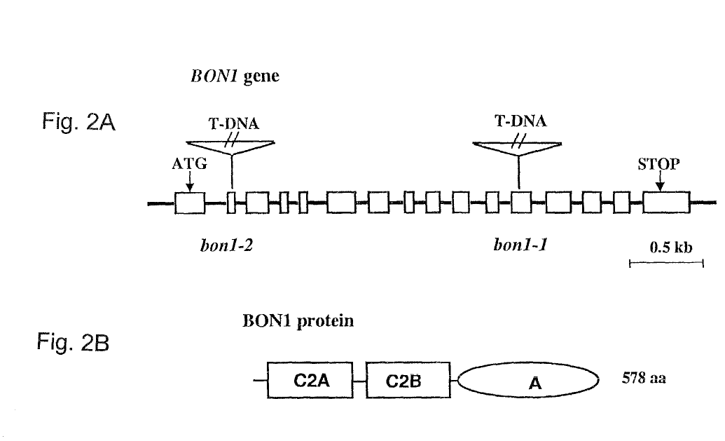

The BON1 gene was cloned on the basis of the T-DNA insertion that results in the bon1 phenotype. The mutation, which segregated as a single recessive trait in the F₂ populations of a backcross to wild-type Columbia, was completely linked to the single T-DNA insertion in this mutant. A 6.5 kb wild-type genomic fragment was isolated, which flanked the T-DNA and a corresponding cDNA of this genomic fragment. Sequence alignment of the genomic fragment and the cDNA indicates that the BON1 gene is comprised of 16 exons and 15 introns (FIG. 2A). The T-DNA is inserted in the twelfth exon of the gene. The bon1-1 mutant defect was complemented either with the genomic fragment or a cDNA-GFP (green fluorescent protein) fusion, confirming the identity of the gene as BON1. Northern blot analysis showed that there was no wild-type RNA transcript of this gene in the mutant, indicating that bon1-1 is a loss-of-function mutant. A second loss-of-function allele bon1-2 (FIG. 2A) was subsequently isolated in the Wassilewskijas background and it exhibited a phenotype similar to that of bon1-1 when introduced into the Columbia background.

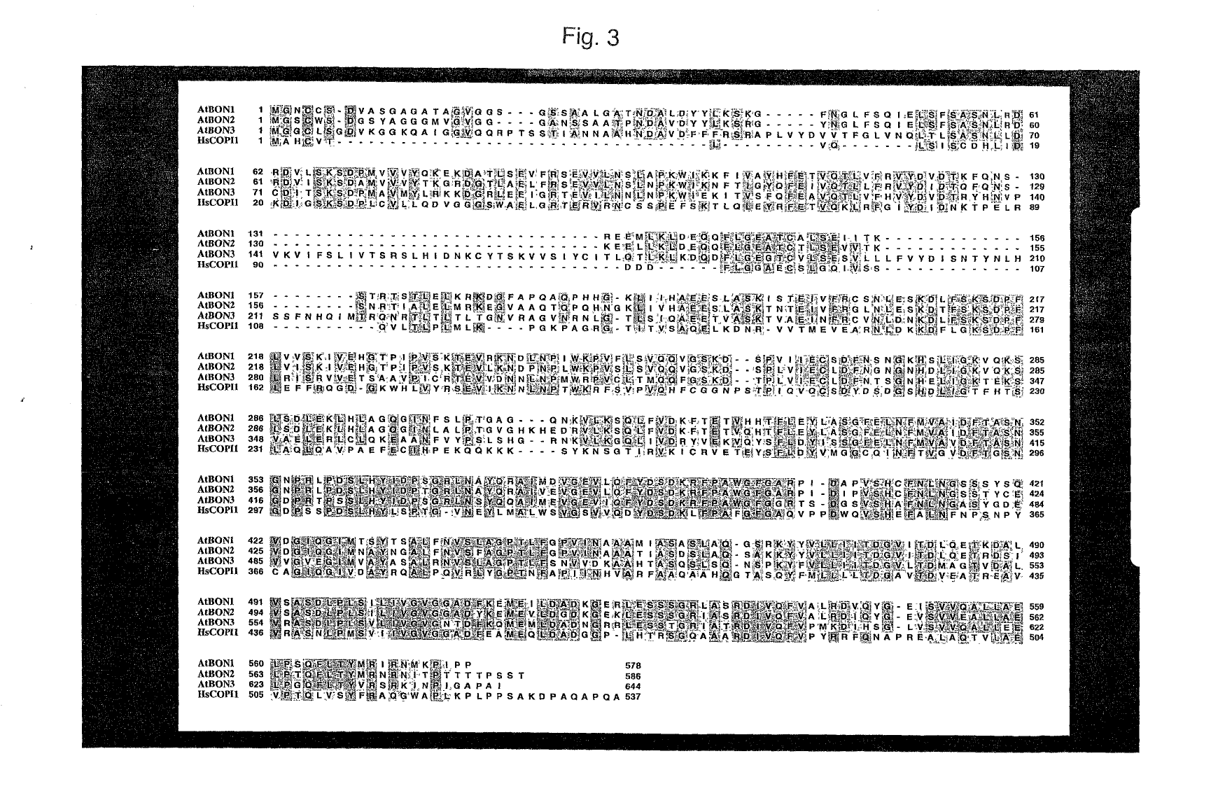

The BON1 gene is predicted to encode a protein of 578 amino acids. The N-terminal portion of the protein consists of two calcium-dependent phospholipid-binding C2 domains (C2A and C2B) and the C-terminal portion exhibits weak similarity to the A domain of integrin (FIG. 2B). The BON1 protein shows extensive homology to the copine gene family. Members of this family have been found in paramecium, worm, mouse, and human and are thought to be calcium-dependent phospholipid binding proteins (Creutz, C. E., J. L. Tomsig et al. (1998) J. Biol. Chem. 273(3):1393-402). In each organism there is more than one member of the gene family in the genome. Arabidopsis thaliana BON1 has two paralogs that have been designated BON2 and BON3. These paralogs are between 72-81% identical and 81-91% similar to each other at the amino acid level. These plant proteins are very similar to their human counterparts. Arabidopsis BON1 has 50% identity and 67% similarity to human copine I over the entire sequence, as shown in FIG. 3.

Analysis of BON1 expression by Northern blots shows that BON1 is expressed at low levels, consistent with its low representation among the population of expression sequence tags (ESTs). Tissue specific Northerns show that expression of BON1 in the leaves and stems is higher than that in roots or flowers, which could explain why leaves and stems are the most severely affected tissues in the bon1-1 mutant.

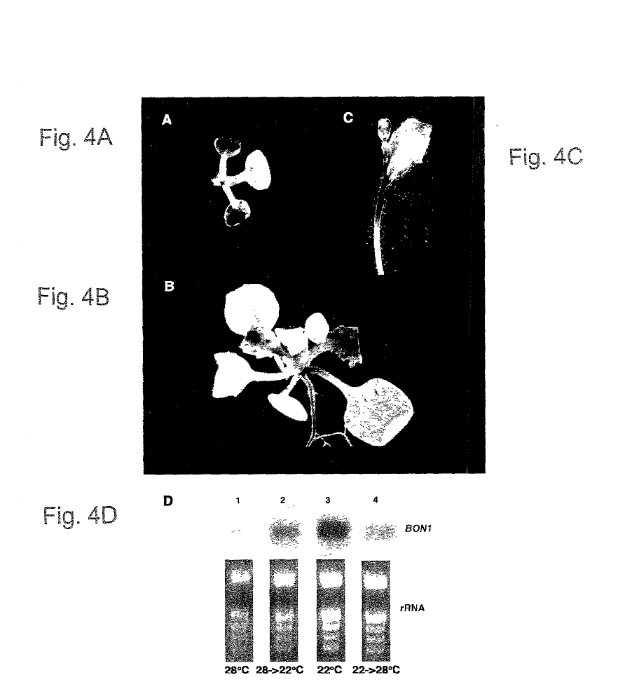

To further analyze the expression of BON1 at the tissue level, a translational fusion of BON1 with a beta-glucuronidase (GUS) reporter gene was made. GUS staining of the BON1-GUS transgenic plants shows that BON1 is more strongly expressed in growing tissues. It was expressed in younger leaves (FIG. 4A) but not in older ones (FIG. 4B). BON1 was also expressed in the inflorescence stems, and was especially concentrated in the apical elongation portion of the stem (FIG. 4C). This expression pattern suggests a direct role of BON1 in regulating cell division and expansion.

The level of expression of BON1 RNA is regulated by temperature, especially during later development. Plants were first grown at one temperature for one month and then half of the population was moved to another temperature for 12 hours. The RNA level of BON1 increased about two-fold when plants were shifted from 28° C. to 22° C. (FIG. 4D, lanes 1 and 2). Conversely, there was a decrease in BON1 RNA level when plants were moved from 22° C. to 28° C. (FIG. 4D, lanes 3 and 4). In plants less than 2 weeks old, BON1 had a relatively high expression level, as compared with older plants. In these plants there was not much difference in the level of BON1 expression between plants grown at 22° C. and those grown at 28° C.

BON1 is a Phospholipid Binding Protein Associated with the Plasma Membrane

The availability of the cloned BON1 gene, coupled with the mutant phenotype, provides an avenue to determine the biological function of a copine gene family member. BON1 contains two C2 domains at the amino-terminus. C2 domains are Ca²⁺ dependent phospholipid-binding domains that confer calcium and/or phospholipid modulation on the activity of the associated domain (Kopka, J., C. Pical et al. (1998) Plant Mol. Biol. 36(5):627-37). In view of these considerations, whether BON1 protein possesses a phospholipid binding activity in vitro was examined.

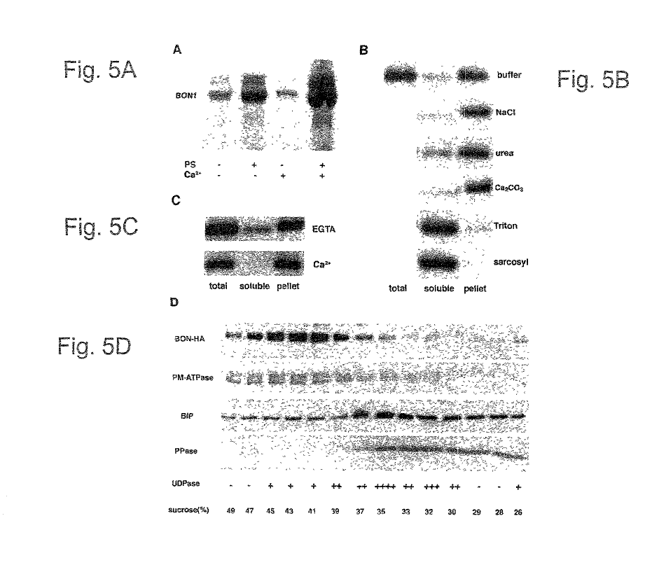

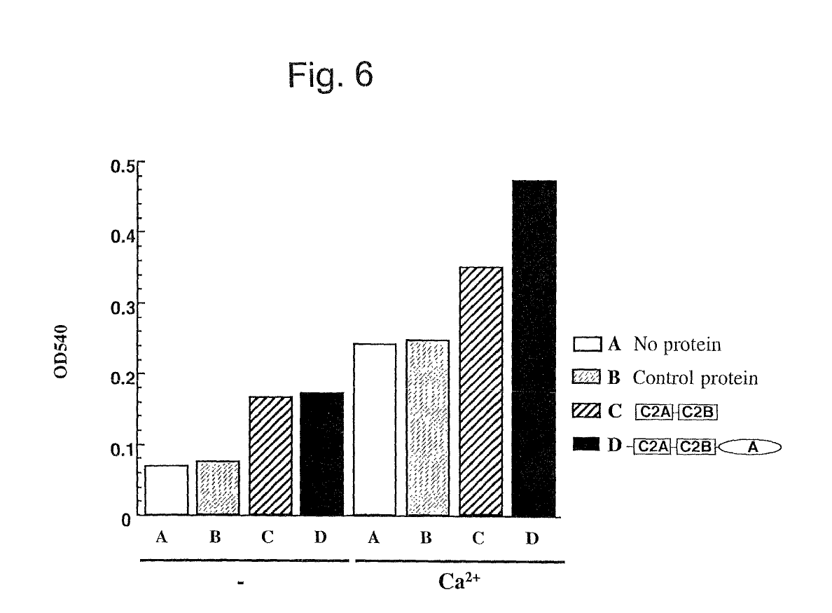

The binding assay utilized a tagged (6× His) BON1 protein that was expressed and purified from E. coli. Purified recombinant BON1 protein was incubated with or without phospholipid and the mixture was pelleted by centrifugation. The proteins associated with the lipids in the pellets were analyzed by electrophoresis on a gel. In the presence of lipid, three times more of BON1 protein was found in the pelleted fraction (FIG. 5A), indicating that BON1 was precipitated by its association with lipid. Addition of calcium to the protein lipid mixture yielded approximately six times more BON1 protein in the pellet (FIG. 5A). This is likely due to an enhanced lipid binding activity of BON1 by calcium or an increased lipid aggregation by calcium which leads to more protein precipitation.

As the association of BON1 with lipids in vitro and its sequence homology suggest a membrane localization, the association of BON1 with membranes in vivo was tested by determining the localization of BON1 using transgenic plants expressing a HA tagged BON1 gene. This BON1-HA gene is functional because it was able to complement the bon1-1 mutant. Total protein was separated into a microsomal fraction and soluble fraction by ultracentrifugation. BON1-HA was found mainly in the microsomal fraction, indicating that it associates with membranes in the cell (FIG. 5B). Several treatments were used to analyze this association. NaCl, urea, or carbonate (pH 111.5) did not alter the distribution of the protein significantly (FIG. 5B). These treatments are capable of stripping peripheral proteins from membranes. Reagents that dissolve membranes such as Triton X-100 and sarcosyl redistributed the protein to the soluble fraction (FIG. 5B). These observations suggest that BON1 is tightly bound to membranes even though the amino acid sequence of the protein does not predict any membrane spanning domains. Although the association of BON1 with membranes was stable in the presence of EGTA (FIG. 5C), the addition of calcium ions partitioned all the BON1 protein to the membrane fraction (FIG. 5C). This indicates that calcium enhances the association of BON1 with membranes although it is not absolutely required for it.

To identify the membrane system with which BON1 is associated, cell fractionation was carried out to separate different endomembranes and the plasma membrane. BON1-HA was not detected in protein extracts from chloroplast, nuclei or cell walls, but was present in the microsomal fraction. The microsomal fraction of BON1-HA plants was separated on a 25% to 50% (w/v) sucrose gradient. The relative position of various membranes on the gradient was determined by assaying fractions for marker proteins specific to each membrane. The BON1-HA protein exhibited a distribution similar to that of the plasma membrane ATPase (FIG. 5D), and distinct from ER, vacuole, and Golgi.

The plasma membrane localization of BON1 indicated by cell fractionation was further supported by analysis in a transient expression system. A BON1-GFP fusion under a strong promoter was electroporated into Arabidopsis protoplasts and the expression of GFP was monitored eight hours later with confocal microscope. The GFP signal was mostly concentrated on the outer membrane of protoplasts, indicating a plasma membrane localization.

BON1 Enhances Vesicle Aggregation in vitro.

One mechanism by which BON1 could stimulate cell expansion and cell division would be to enhance membrane trafficking by facilitating the association and fusion of vesicles with the membrane. The ability of BON1 to enhance vesicle aggregation in vitro was tested using an assay that measures the turbidity of a lipid solution. Aggregation of the lipid vesicles is accompanied by an increase in turbidity (monitored by the absorbance at 540 nm). Constructs encoding the full-length BON1 protein, the N-terminal 2C2 domain, and the C-terminal A domain each with 6× His tags were expressed in E. coli and the resulting His-tagged proteins were purified. These recombinant proteins were incubated with phosphotidyl serine (PS) or phosphotidyl inositol (PI). A difference in turbidity was observed with PS but not PI. Both the full length BON1 protein and the 2C2 domain increased the absorbance of PS solution (FIG. 6), indicative of an activity that promotes vesicle aggregation. By contrast, the A domain of the BON1 protein had no effect on the turbidity (FIG. 6). As has been previously observed (Creutz, C. E., J. L. Tomsig et al. (1998) J. Biol. Chem. 273(3):1393-402), addition of calcium alone stimulates the aggregation of vesicles. Addition of calcium in the presence of either the 2C2 domain or the full-length BON1 enhanced the aggregation considerably over the background stimulation observed in the presence of calcium (FIG. 6).

BAP1 is a BON1 Interacting Protein.

The data indicate that BON1 functions to promote vesicle trafficking during cell expansion and division. It is likely that BON1 does so via its interaction with other proteins, so the yeast two hybrid assay was used to look for proteins that interact with BON1. The A domain (from Val³¹⁹ to Pro⁵⁷⁸) was used as bait because it is the most likely segment of BON1 to be involved in protein-protein interactions (Creutz, C. E., J. L. Tomsig et al. (1998) J. Biol. Chem. 273(3):1393-402). A cDNA library made from the vegetative tissues of 3-week-old plants was screened for clones that signaled an interaction with this bait. Twenty-three positive clones rescued from the screen are the same gene, which was called the BON1 Association Protein1 (BAP1).

BAP1 encodes a protein of 192 amino acids. The two-hybrid clone comprises the entire BAP1 except for the first 6 amino acids. A blast search reveals that it has homology to another putative Arabidopsis protein in the database which, for purposes of this study, was called BAL:BAP1 Like. SMART (Simple Modular Architecture Research Tool) search indicates that the amino-terminal part of BAP1 (approximately 120 amino acids) has sequence homology and structural analogy to the C2 domain. C2 domain family is very divergent (Rizo, J., T. C. Sudhof (1998) J. Biol. Chem. 273(26):15879-82), and the C2 domain of BAP1 is not significantly homologous to those of BON1 at the protein sequence level. The C-terminal 52 amino acids of BAP1 is 54% identical to those of BAL, but does not show significant homology to any known motifs.

BAP1 Has a Similar Function to BON1.

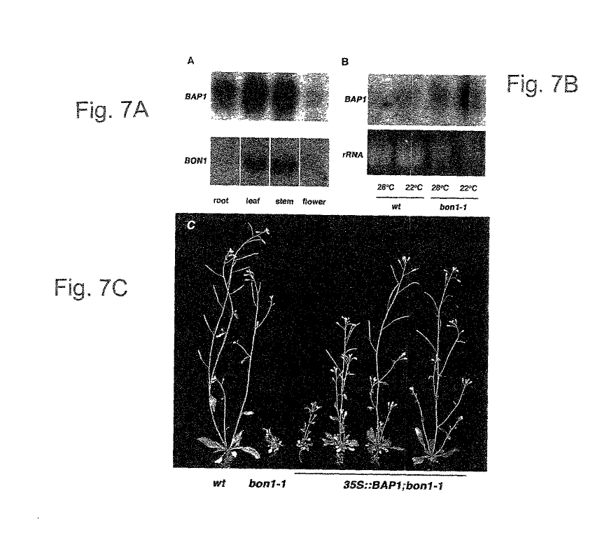

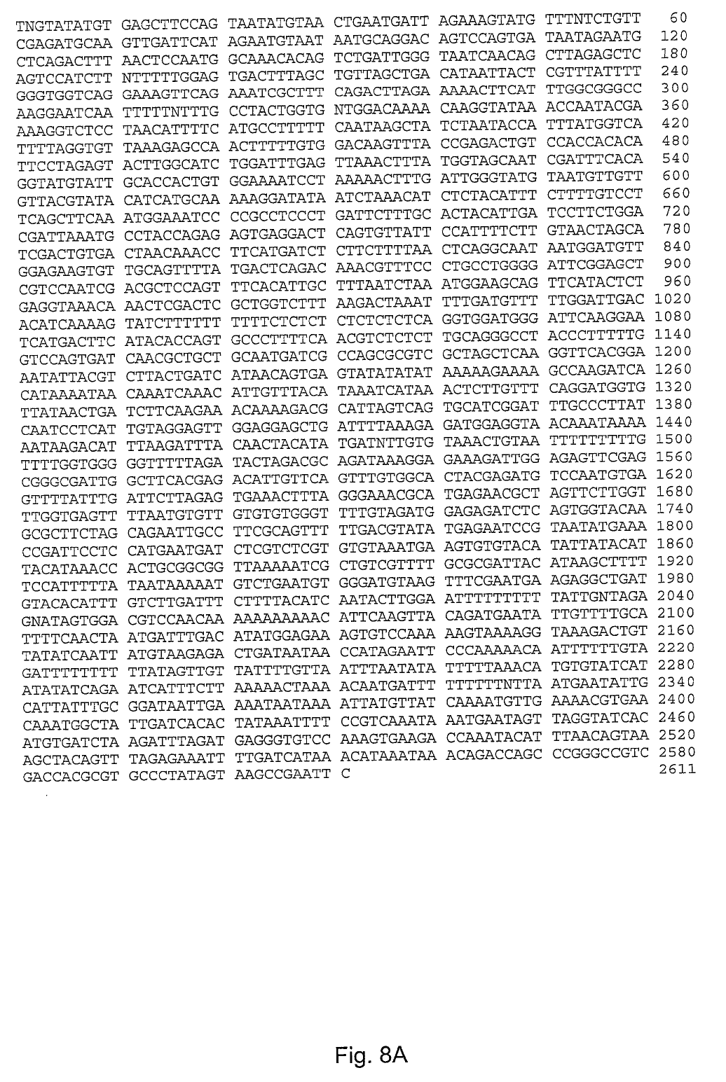

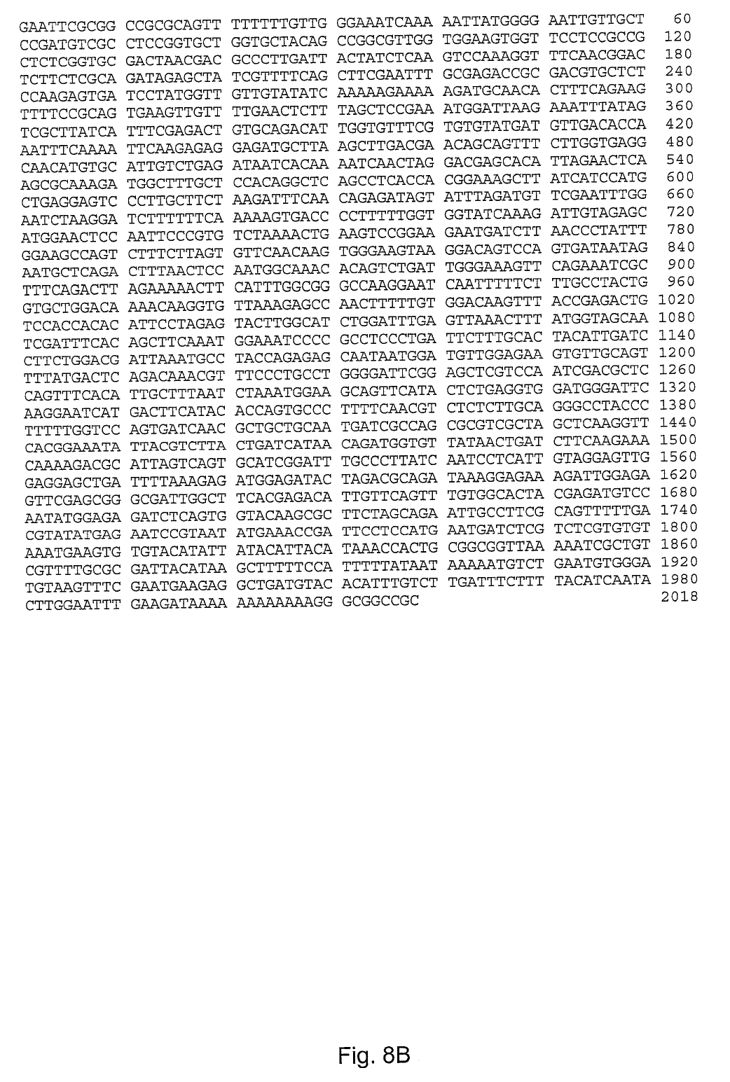

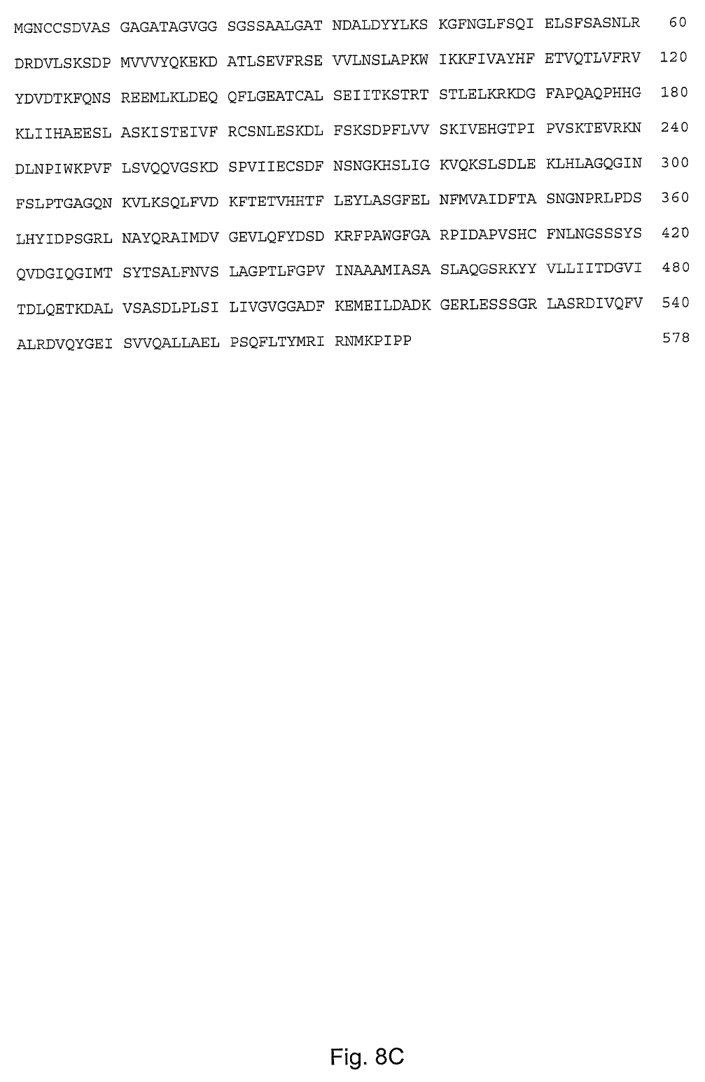

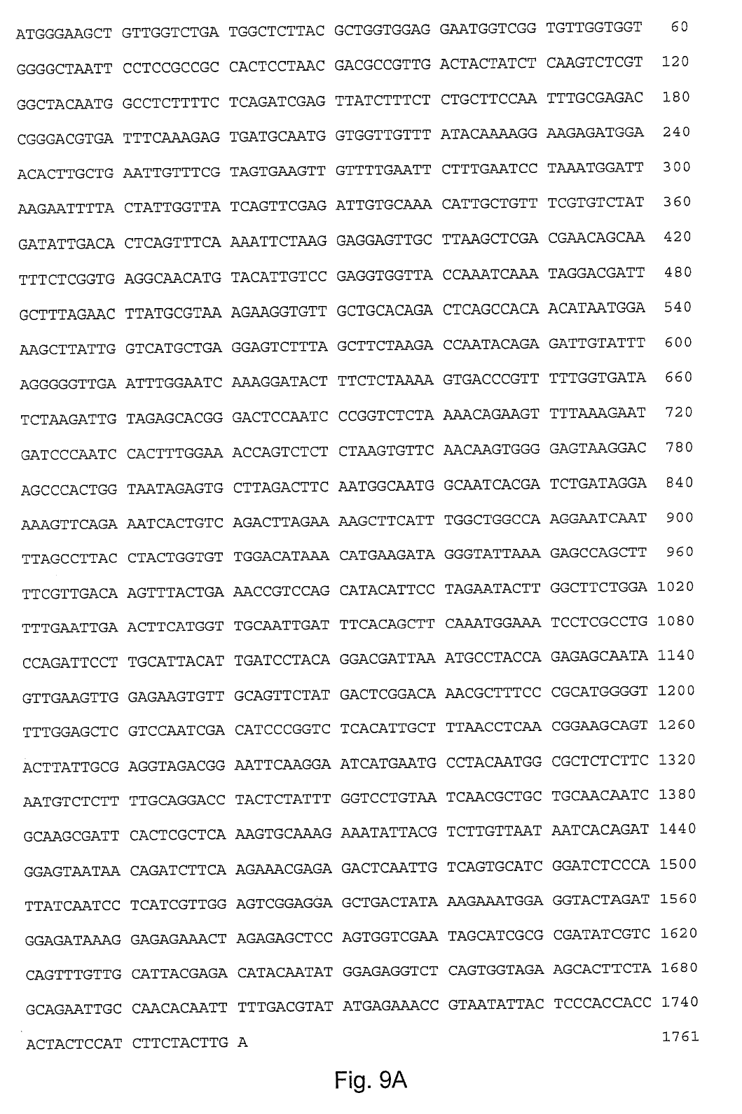

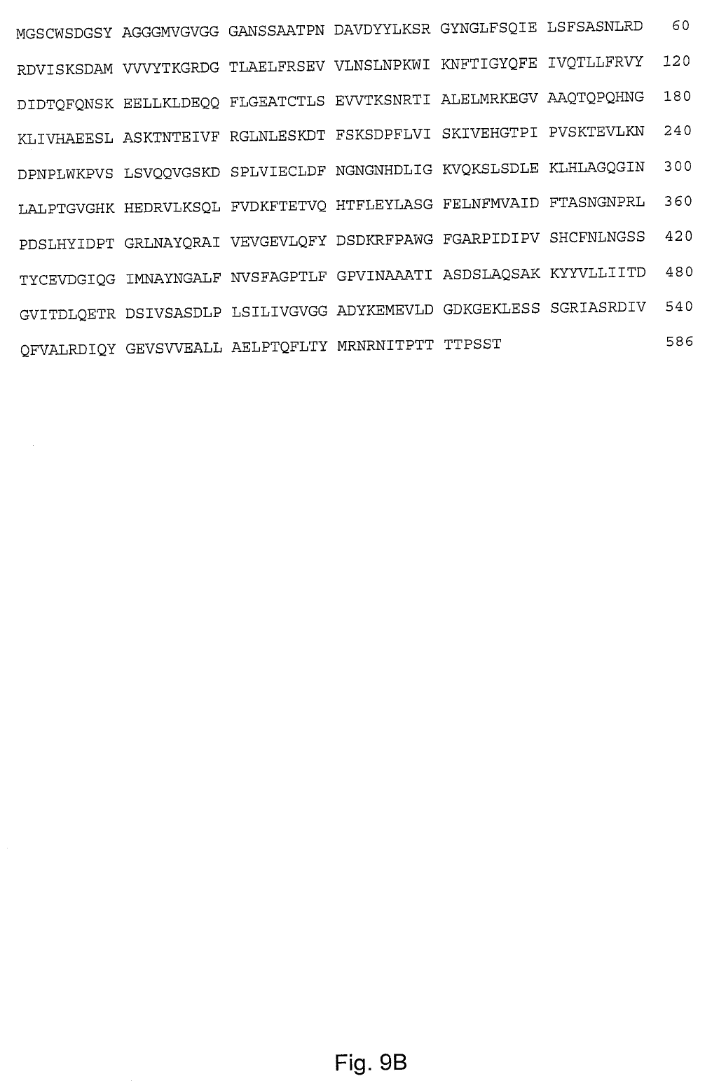

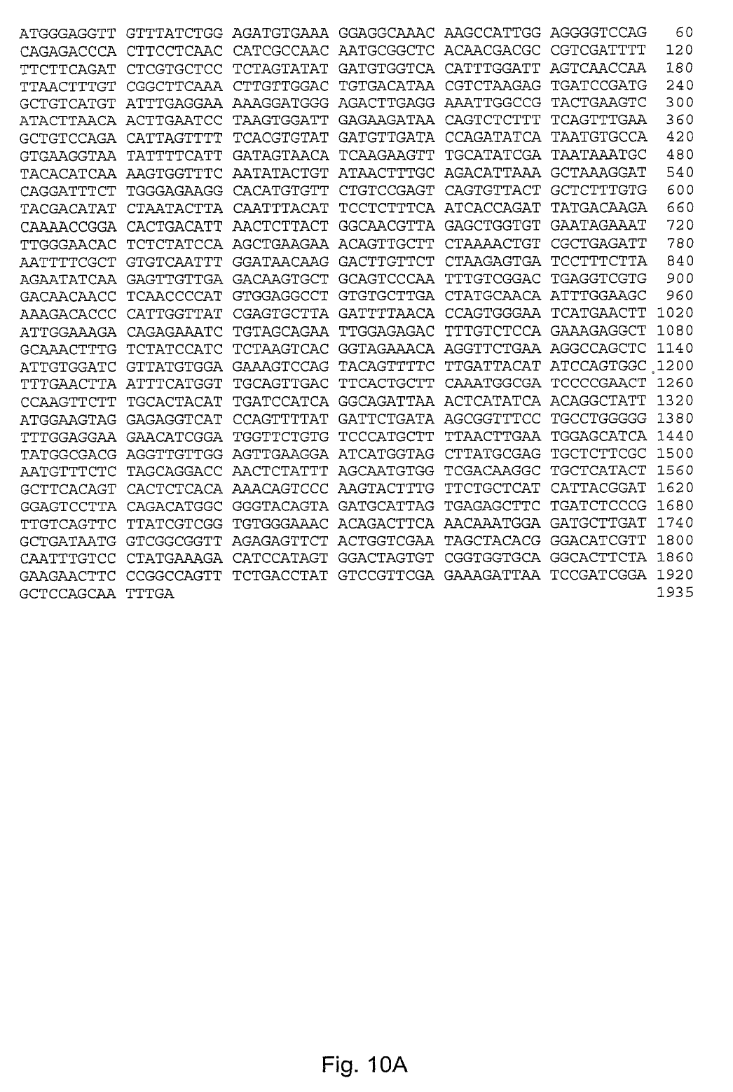

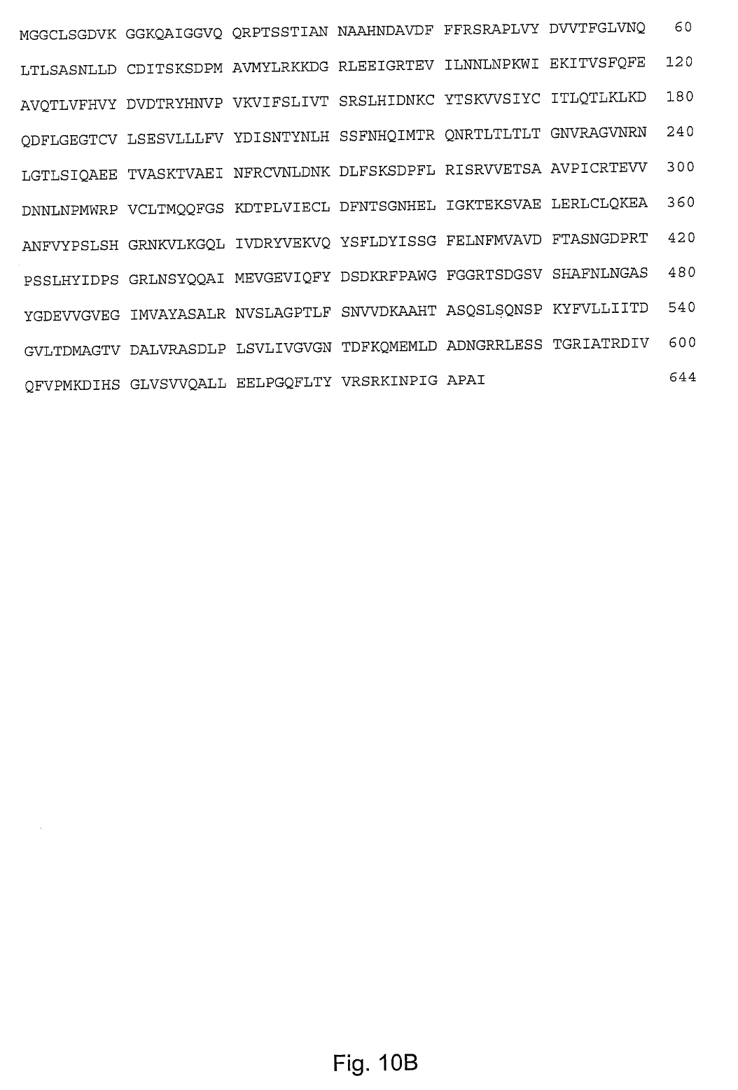

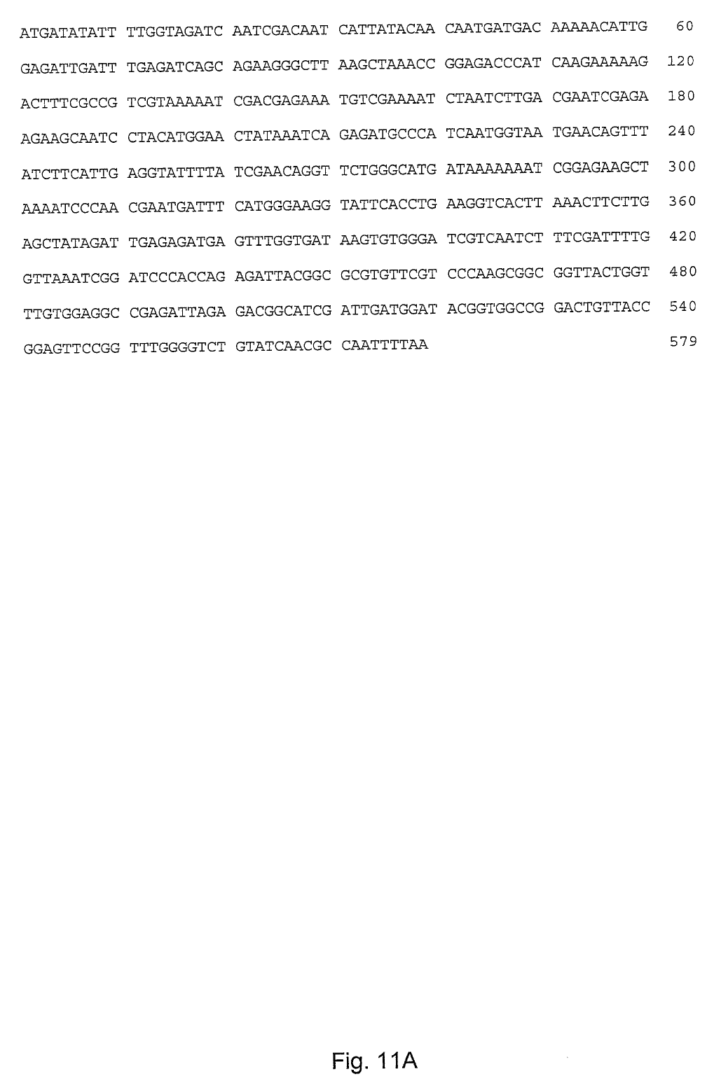

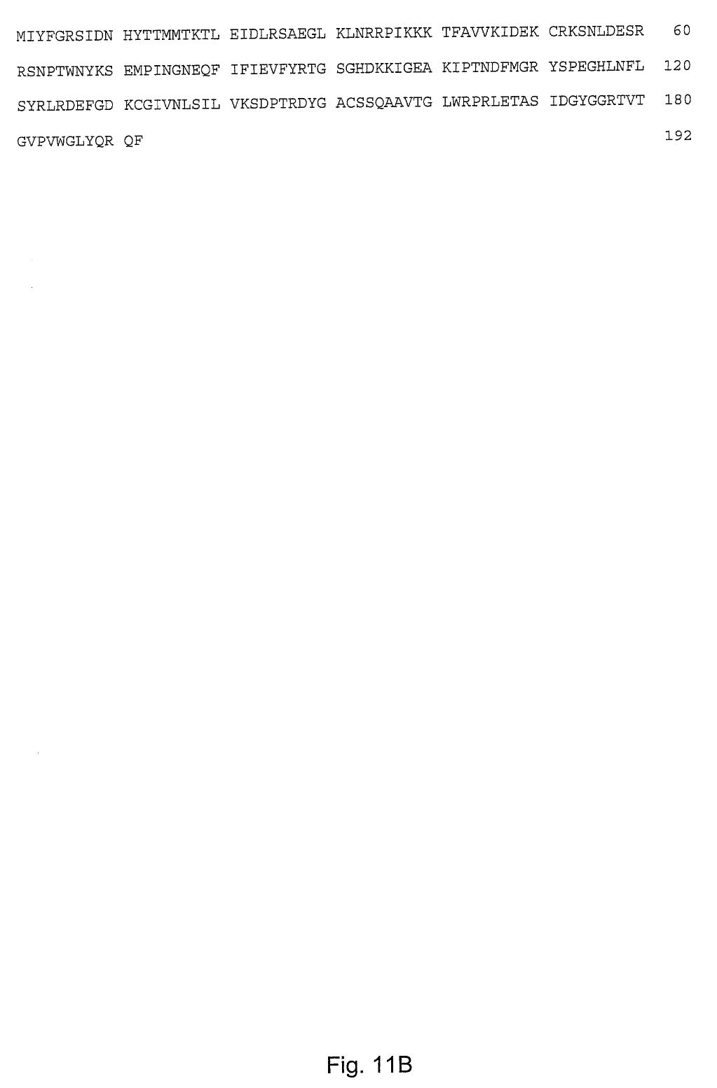

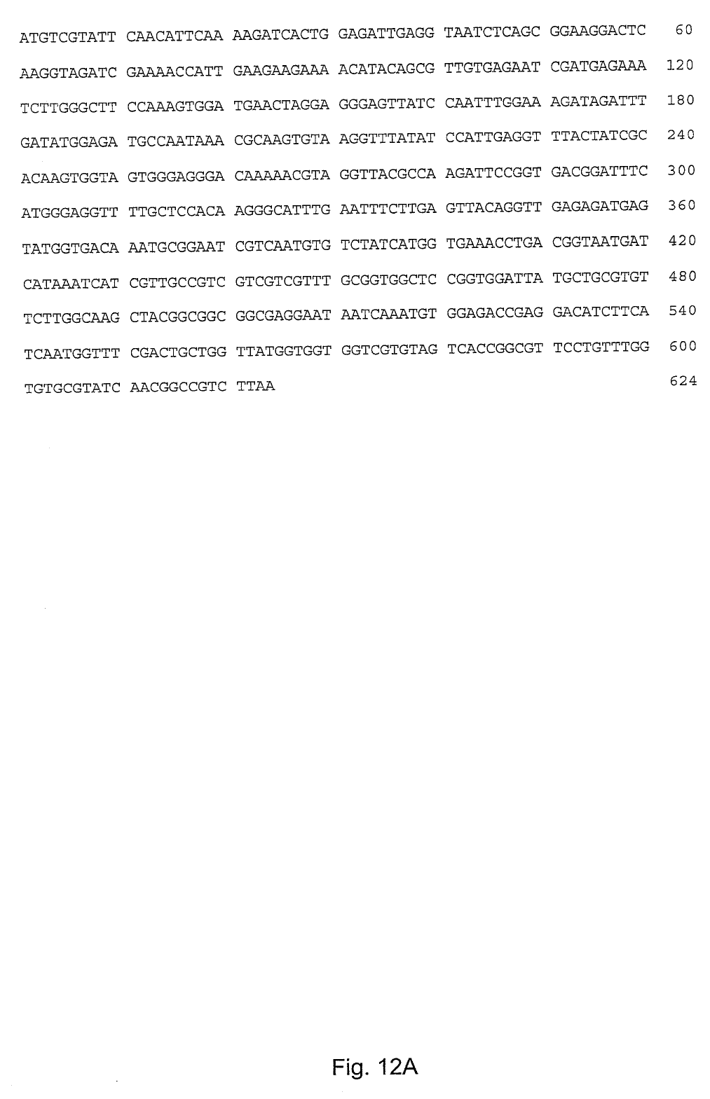

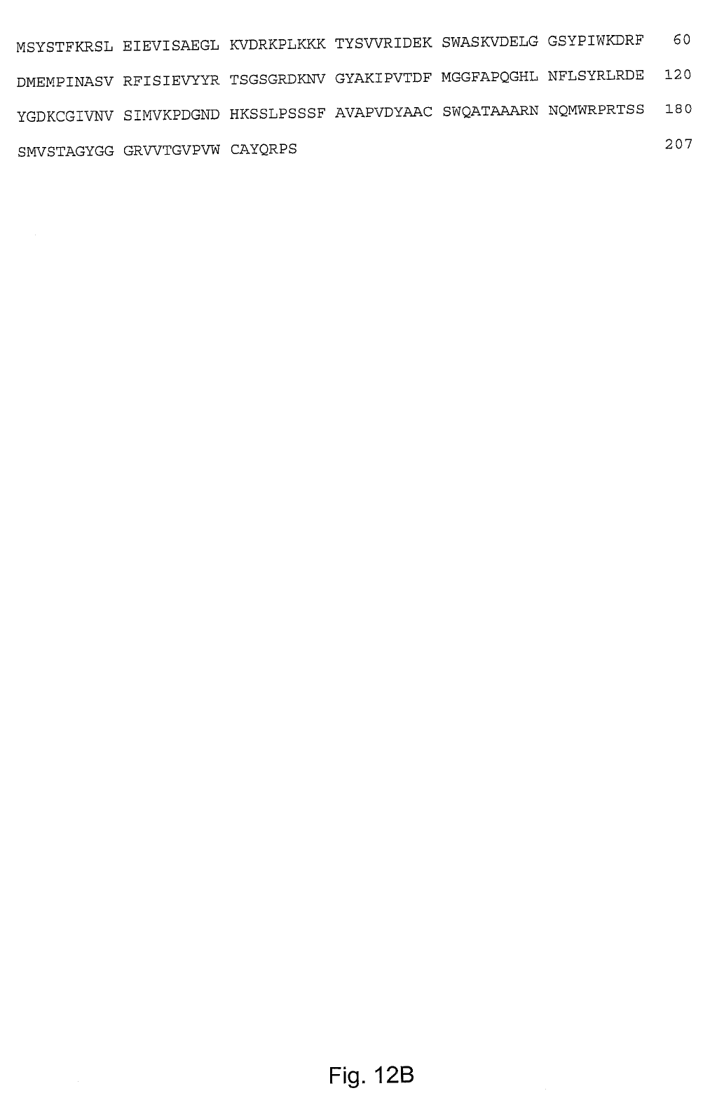

BAP1 is expressed ubiquitously throughout the roots, leaves, stems, and flowers with expression in leaves and stems relatively higher than in other parts of the plant, and BAP1 also has higher expression in roots (FIG. 7A). The expression of BAP1, like that of BON1, is modulated by temperature. There was an increase in BAP1 RNA when plants were shifted from higher to lower temperature (FIG. 7B). Conversely, higher temperature repressed BAP1 expression (FIG. 7B). Moreover, BAP1 expression is affected by BON1. In the bon1-1 mutant, there was more BAP1 transcript and the modulation of its expression by temperature was more pronounced than in wild type (FIG. 7B).

To identify whether BAP1 has a similar function to that of BON1, the BAP1 gene was expressed under the CaMV 35S promoter and the transgene was introduced into the bon1-1 mutant. If the two proteins are involved in the same event, then overexpression of BAP1 would likely suppress the mutant defects of the bon1 mutant. Of 18 transgenic lines analyzed, nine showed suppression to varying degrees, ranging from good (1), moderate (3), to weak (5) (FIG. 7C). Those plants that were partially suppressed had more elongated stems and more expanded leaves than the bon1 mutant. Thus overexpression of BAP1 can suppress the defect in bon1, but the extent of suppression may be modified by the site of transgene integration or transgene silencing.

Discussion

Loss of BON1 function leads to miniature plants at ambient temperature, due to a reduction in both cell expansion and cell division, but has no obvious phenotype at high temperature.

Overexpression of the Arabidopsis BAP1 gene partially suppresses the bon1 phenotype, establishing a functional connection between these two genes.

The bon1 phenotype differs from that of a typical cold sensitive mutation in a vital gene. Null mutations in a vital gene would be lethal at all temperatures, whereas BON1 function and BON1 protein are required to maintain normal plant size at low temperature. Mutants with a conditional cold-sensitive phenotype similar to that of bon1 have been identified previously (Tsukaya, H., S. Naito et al. (1993) Development 118:751-764; Akamatsu, T., Y. Hanzawa et al. (1999) Plant Physiol. 121(3):715-22). These mutants, acaulis1, acaulis3 and acaulis4, or acl1, acl3, and acl4, respectively) grow normally at 28° C. and have reduced stem elongation and leaf expansion at the lower temperature. It is possible that they could be involved in the same genetic pathway as BON1 or in a parallel pathway that maintains size at low temperature. However, such conclusions are premature because neither the identity of the genes nor the nature of the mutations responsible for these phenotypes are known.

Another Arabidopsis mutant, with increased levels of stearate, also exhibits a miniature phenotype at 22° C., which can be suppressed only at the very high temperature of 36° C. (Lightner, J., D. W. J. James et al. (1994) Plant J. 613):401-412).

Further support for the role of BON1 and BAP1 in homeostasis at low temperature comes from analysis of the transcriptional pattern of the two genes. First, BON1 is expressed more strongly in growing tissues than in mature tissues. Second, both BON1 and BAP1 expression are under temperature control in that both have elevated expression when plants are shifted from high to lower temperature. Moreover, the enhancement of BAP1 expression in response to low temperature is more drastic in the bon1 background than in the wild type. These data indicate that BON1 and BAP1 are required for normal growth at lower temperature and that they are upregulated under the conditions where their function is required. There appears to be an additional compensatory stimulation of BAP1 expression when there is insufficient BON1.

Study of BON1 provides important insights into the function of the copine gene family, of which BON1 is a member. The copine family includes members from protozoa to humans, but despite its evolutionary conservation, little is known about the function of its members. Previous work in paramecium and mice have shown that copines have lipid binding activity and promote the aggregation of vesicles (Creutz, C. E., J. L. Tomsig et al. (1998) J. Biol. Chem. 273(3):1393-402). It was suggested that the paramecium copine might be involved in vesicle trafficking because of its association with secretory vesicles (Creutz, C. E., J. L. Tomsig et al. (1998) J. Biol. Chem. 273(3):1393-402). In mice the N-copine expressed in the hippocampus is induced by kainate stimulation. The protein is detected in neurons, both in the cell bodies and dendrites (Nakayama, T., T. Yaoi et al. (1999) J. Neurochem. 72(1):373-9). Kainate has been reported to stimulate long-term potentiation and it was therefore surmised that N-copine has a role in synaptic plasticity (Nakayama, T., T. Yaoi et al. (1998) FEBS Lett. 428(1-2):80-4).

The data described herein indicates that BON1 protein likely regulates membrane biogenesis and cell wall remodeling through exocytosis, which ultimately controls cell size. All copines have a similar domain structure: the C2 amino terminal domain and a carboxyl-terminal A domain. Like the paramecium and mice copines, the full length Arabidopsis BON1 protein and the truncated C2 domain bind phospholipids and the binding is stimulated by calcium. Furthermore, BON1 promotes aggregation of lipid vesicles in vitro. Therefore, all copines may have similar biochemical activities in addition to structural similarities. Previous work provided only a few hints as to the subcellular localization of copines. N-copine was localized to postsynaptic membranes where synaptic plasticity occurs (Nakayama, T., T. Yaoi et al (1999) J. Neurochem. 72(l):373-9) and one of the chromaffin granule-binding proteins (chromobindin 17) is likely to be a copine (Creutz, C. E., J. L. Tomsig et al. (1998) J. Biol. Chem. 273(3):1393-402). The data herein show that BON1 protein is mainly associated with the plasma membrane.

It seems likely that the copine BON1 and its interacting protein BAP1 function directly in fusion of vesicles with plasma membrane at low temperature, which is a process that contributes both to membrane growth and wall expansion. Exocytosis is required for cell growth in many organisms and in plant cells a decrease in exocytosis has been shown to decrease cell size (Carroll, A. D., C. Moyen et al. (1998) Plant Cell 10(8): 1267-76). In other systems, the core machinery for vesicle docking and fusion has been identified (Jahn, R. and T. C. Sudhof (1999) Annu. Rev. Biochem. 68:863-911). However it is not well understood how the basic fusion machinery is regulated. As fusion is a temperature-dependent process (Weber, T., B. V. Zemelman et al. (1998) Cell 92(6):759-72), the copine/BAP1 proteins may be required to accelerate a process that occurs less rapidly at low temperature. Indeed, some of the mutations defective in membrane fusion are cold-sensitive in yeast (Lehman, K., G. Rossi et al. (1999) J. Cell Biol. 146(1):125-40; Otte, S., W. J. Belden et al. (2001) J. Cell Biol. 152(3):503-518).

It is likely that BON1 functions in temperature homeostasis either by acting catalytically (increasing the fusion of vesicles with the membrane) or structurally (by associating with the plasma membrane to maintain membrane function at low temperature). Recently, a protein kinase activity was attributed to the A domain of human copine m (Caudell, E. G., J. J. Caudell et al. (2000) Biochem. 39(42):13034-43) suggesting that these proteins have enzymatic function. Copines in other organisms may function similarly to accelerate vesicle trafficking in response to specific environmental signals. For example, the neuronal copines could accelerate membrane trafficking at the synapse upon chemical stimulation thereby enhancing synaptic transmission.

It is clear, however, that the acl1, acl3 and al4 mutants are not identical to the BON1. In acl1 mutants (Tsukaya, H., S. Naito et al. (1993) Development 118:751-764), the length of the cells is less than that of wild type, as in bon1 mutants, but unlike the bon1 mutants, the number of cells in the leaves and internodes is the same as in wild type plants. In bon1, the number, as well as the size of the cells, is decreased relative to wild type plants. In addition, the acl1 mutants exhibit cessation of development of the inflorescence meristems at 22° C., whereas the meristems in bon1 mutants grow shorter than wild type, but do not cease growth. The acl3 and acl4 mutants were mapped to chromosomes 3 and 4, respectively (Akamatsu, T., Y. Hanzawa et al. (1999) Plant Physiol. 121(3):715-22). BON1 shows some similarity to GenBank accession AB022212, which is located onArabidopsis chromosome 5. BAP1 shows some similarity to GenBank accession AL137898, which is located on Arabidopsis chromosome 3.

### Example 2

### Transgenic Plants with BON1-GUS Fusion Gene Have bon1 Loss-of-Function Phenotype

The full-length BON1 gene was translationally fused with a reporter gene GUS and transformed to wild type Arabidopsis. The transformants exhibited bon1-1 phenotype in that they were miniature plants with small curly leaves and short stems, very similar to bon1-1. Furthermore, this phenotype was exhibited only at 22° C., but not at 28° C., as was bon1-1. Therefore, the BON1-GUS chimeric gene acts either in a dominant negative fashion or induces cosuppression in wild type plants.

This finding provides a method to generate miniature plants in other species. The technique to make knockout plants through homologous recombination is not available in any higher plants. It is therefore not possible to make a bon1 mutant in other plant species via homologous recombination. However, transformation techniques are readily available in many plant species, allowing introduction of the BON1-GUS transgene, antisense constructs, or other BON1 chimeras into other species to make miniature plants.

### Example 3

### Transgenic Plants with BAP1-GUS Fusion Gene Have Dwarf Phenotype

The N-terminal two-thirds of the BAP1 gene was translationally fused with GUS gene and was introduced into wild-type Arabidopsis. The BamHI site in the BAP1 protein, the restriction site immediately after Thr¹⁴³, was used for the fusion.

The transgenic plants exhibited dwarf phenotype and some exhibited arrested growth, which led to death before flowering. The phenotype is reminiscent to that of the bon1-1 mutant. Interestingly, this phenotype was temperature dependent, and was present at lower temperature, but absent at higher temperature.

This finding shows that BAP1 acts closely with BON1 in plants. BON1 and BAP1 are involved in the same process of maintaining growth homeostasis at lower temperature.

### Example 4

### Overexpression of BON1

The BON1 gene was expressed under the control of the CaMV 35S promoter and transformed into wild type Arabidopsis, as described above. Approximately ⅕ of the transformants exhibited larger leaves and thicker stems than equivalent wild type plants, and the increase in size appeared to be due to an increase in both cell size and cell number. Overexpression of BON1 therefore appears to result in increased growth and larger plants as compared to wild type.

### Example 5

### Overexpression of BAP1

The BAP1 gene was expressed under the control of the CaMV 35S promoter and transformed into wild type Arabidopsis. Approximately 50% of the transformants exhibited larger leaves and more extensive root growth than equivalent wild type plants. Overexpression of BAP1 therefore appears to result in increased growth and larger plants as compared to wild type.

### Example 6

### Expression in Maize Protoplasts

Maize plants were grown in the dark until the leaves were 10 cm long. The leaves were cut into 0.5 mm strips, digested in cellulase and macerozyme in a shaking flask. The protoplasts were then purified. The BON1-GFP fusion, prepared as described above, was introduced into the maize protoplasts by electroporation. The chimeric protein was expressed in plasma membrane, as it was expressed in Arabidopsis protoplasts.

All patent, patent applications and references are incorporated herein in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated nucleic acid comprising SEQ ID NO:2, and degenerate variants thereof.

2. An isolated nucleic acid comprising 315 or more consecutive nucleotides of SEQ ID NO:2.

3. An isolated nucleic acid comprising a sequence that encodes a polypeptide having an amino acid sequence which is 94% identical to SEQ ID NO:3.

4. An isolated polypeptide comprising the amino acid sequence of SEQ ID NO:3.

5. An isolated polypeptide comprising 400 or more consecutive amino acid residues of SEQ ID NO:3.

6. An isolated polypeptide comprising a sequence that has 94% sequence identity to SEQ ID NO:3.

7. A vector comprising the isolated nucleic acid of claim 1.

8. An isolated cell comprising the vector of claim 7.

9. The cell of claim 8, wherein the cell is selected from the group consisting of: a prokaryotic cell and a eukaryotic cell.

10. A transgenic plant which has an altered size compared to a corresponding non-transgenic plant, comprising introducing into the plant exogenous nucleic acid which modulates BON1 in the plant.

11. A transgenic plant that is smaller in size than a corresponding non-transgenic plant, comprising introducing exogenous nucleic acid which inhibits BON1 in the plant.

12. The transgenic plant of claim 11, wherein the exogenous nucleic acid is inserted in the twelfth exon of the BON1 gene.

13. The transgenic plant of claim 12, wherein the exogenous nucleic acid is a fusion of BON1 with a beta-glucuronidase gene.

14. The transgenic plant of claim 11, wherein the exogenous nucleic acid is inserted in the second exon of the BON1 gene.

15. A transgenic tissue culture derived from the transgenic plant of claim 11.

16. A transgenic seed of the transgenic plant of claim 11.

17. A transgenic plant, plant part, plant cell or tissue culture, grown from the transgenic seed of claim 16.

18. The transgenic plant, plant part, plant cell or tissue culture of claim 17, wherein the plant is an ornamental plant or a turfgrass, or the plant part, plant cell or tissue culture is derived from an ornamental plant or a turfgrass.

19. A transgenic plant that is larger in size than a corresponding non-transgenic plant, comprising introducing into the plant an exogenous nucleic acid which enhances BON1 in the plant.

20. The transgenic plant of claim 19, wherein the exogenous nucleic acid is an exogenous BON1 gene.

21. A method of producing a transgenic plant which has an altered size compared to a corresponding non-transgenic plant, comprising introducing into the plant exogenous nucleic acid which modulates BON1 in the plant.

22. A method of producing a transgenic plant that is smaller in size than a corresponding non-transgenic plant, comprising introducing into the plant an exogenous nucleic acid which inhibits BON1 in the plant.

23. A method of producing a transgenic plant that is smaller in size than a corresponding non-transgenic plant, comprising mutating the endogenous BON1 in the plant.

24. The method of claim 22, wherein the exogenous nucleic acid is a fusion of BON1 with a beta-glucuronidase gene (BON1-GUS).

25. The method of claim 22, wherein the nucleic acid results in overexpression of the C-terminus of a BON1.

26. The method of claim 22, wherein the nucleic acid results in overexpression of the full length BON1.

27. The method of claim 22, wherein the transgenic plant is selected from the group consisting of: angiosperms and gymnosperms.

28. The method of claim 22, wherein the transgenic plant is an ornamental plant or a turfgrass.

29. A transgenic plant produced by the method of claim 22.

30. A method of producing a transgenic plant that is larger in size than a corresponding non-transgenic plant, comprising introducing into the plant an exogenous nucleic acid which enhances BON1 in the plant.

31. The method of claim 30, wherein the exogenous nucleic acid is an exogenous BON1 gene.

32. The method of claim 30, wherein the plant is a crop plant.

33. The method of claim 30, wherein the plant is a biomass plant.

34. A transgenic plant produced by the method of claim 30.

35. A method of modulating homeostasis of a plant, the method comprising introducing into the plant an exogenous nucleic acid which modulates BON1 in the plant.

36. A method of increasing the yield of a plant, comprising introducing into the plant an exogenous nucleic acid which enhances BON1 in the plant.

37. A method of producing a transgenic plant that is able to grow at a higher altitude or in a lower temperature region than a corresponding non-transgenic plant, comprising introducing into the plant an exogenous nucleic acid that enhances BON1 in the plant.

38. An isolated nucleic acid comprising SEQ ID NO:5, and degenerate variants thereof.

39. An isolated nucleic acid comprising 170 or more consecutive nucleotides of SEQ ID NO:5.

40. An isolated nucleic acid comprising a sequence that encodes a polypeptide having an amino acid sequence which is 97% identical to SEQ ID NO:6.

41. An isolated polypeptide comprising the amino acid sequence of SEQ ID NO:6.

42. An isolated polypeptide comprising 550 or more consecutive amino acid residues of SEQ ID NO:6.

43. An isolated polypeptide comprising a sequence that has 97% sequence identity to SEQ ID NO:6.

44. A vector comprising the isolated nucleic acid of claim 38.

45. An isolated cell comprising the vector of claim 45.

46. The cell of claim 45, wherein the cell is selected from the group consisting of: a prokaryotic cell and a eukaryotic cell.

47. An isolated nucleic acid comprising SEQ ID NO:11, and degenerate variants thereof.

48. An isolated nucleic acid comprising 390 or more consecutive nucleotides of SEQ ID NO:11.

49. An isolated nucleic acid comprising a sequence which has 99% sequence identity to the coding sequence of SEQ ID NO:11.

50. An isolated nucleic acid comprising a sequence that encodes a polypeptide having an amino acid sequence which is 98% identical to SEQ ID NO:12.

51. An isolated polypeptide comprising the amino acid sequence of SEQ ID NO:12.

52. An isolated polypeptide comprising 180 or more consecutive amino acid residues of SEQ ID NO:12.

53. A vector comprising the isolated nucleic acid of claim 47.

54. An isolated cell comprising the vector of claim 53.

55. The cell of claim 54, wherein the cell is selected from the group consisting of: a prokaryotic cell and a eukaryotic cell.

56. A method of producing a transgenic plant which has an altered size compared to a corresponding non-transgenic plant, comprising introducing into the plant exogenous nucleic acid which modulates BAP1 in the plant.

57. A method of producing a transgenic plant that is smaller in size than a corresponding non-transgenic plant, comprising introducing into the plant an exogenous nucleic acid which inhibits BAP1 in the plant.

58. The method of claim 57, wherein the nucleic acid results in overexpression of the C-terminus of a BAP1.

59. The method of claim 57, wherein the transgenic plant is selected from the group consisting of: angiosperms and gymnosperms.

60. The method of claim 57, wherein the transgenic plant is an ornamental plant or a turfgrass.

61. A transgenic plant produced by the method of claim 57.

62. A method of producing a transgenic plant that is larger in size than a corresponding non-transgenic plant, comprising introducing into the plant an exogenous nucleic acid which enhances BAP1 in the plant.

63. The method of claim 62, wherein the exogenous nucleic acid is an exogenous BAP1 gene.

64. The method of claim 62, wherein the plant is a crop plant.

65. The method of claim 64, wherein the plant is a biomass plant.

66. A transgenic plant produced by the method of claim 62.

67. A method of increasing the yield of a plant, comprising introducing into the plant an exogenous nucleic acid which enhances BAP1 in the plant.

68. A method of producing a transgenic plant that is able to grow at a higher altitude or in a lower temperature region than a corresponding non-transgenic plant, comprising introducing into the plant an exogenous nucleic acid that enhances BAP1 in the plant.
